SEQUENCE LISTING (10 sequences)

SEQ ID NO: 1 - DNA, 20 bp, synthetic construct
  source 1..20: mol_type = other DNA
ccagcccccaccagtccact

SEQ ID NO: 2 - DNA, 20 bp, synthetic construct
  source 1..20: mol_type = other DNA
gctgcatgatctccttggcg

SEQ ID NO: 3 - DNA, 20 bp, synthetic construct
  source 1..20: mol_type = other DNA
agatagctggtgacagacag

SEQ ID NO: 4 - DNA, 20 bp, synthetic construct
  source 1..20: mol_type = other DNA
cgtggagagatcatctgagg

SEQ ID NO: 5 - DNA, 20 bp, synthetic construct
  source 1..20: mol_type = other DNA
tcgaaaaggtcaagtgctac

SEQ ID NO: 6 - DNA, 20 bp, synthetic construct
  source 1..20: mol_type = other DNA
tggtgcagcggcagcggcag

SEQ ID NO: 7 - DNA, 20 bp, synthetic construct
  source 1..20: mol_type = other DNA
ggcgcgagcgcgggcctagc

SEQ ID NO: 8 - protein, 342 aa, Homo sapiens
GAMDPRVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKXFCGTPEYLAPEVLEDNDYGRAVDWWGLGVVMYEMMCGRLPFYNQDHEKLFELILMEEIRFPRTLGPEAKSLLSGLLKKDPKQRLGGGSEDAKEIMQHRFFAGIVWQHVYEKKLSPPFKPQVTSETDTRYFDEEFTAQMITITPPDQDDSMECVDSERRPHFPQFDYSASSTA

SEQ ID NO: 9 - protein, 481 aa, Homo sapiens
MNEVSVIKEGWLHKRGEYIKTWRPRYFLLKSDGSFIGYKERPEAPDQTLPPLNNFSVAECQLMKTERPRPNTFVIRCLQWTTVIERTFHVDSPDEREEWMRAIQMVANSLKQRAPGEDPMDYKCGSPSDSSTTEEMEVAVSKARAKVTMNDFDYLKLLGKGTFGKVILVREKATGRYYAMKILRKEVIIAKDEVAHTVTESRVLQNTRHPFLTALKYAFQTHDRLCFVMEYANGGELFFHLSRERVFTEERARFYGAEIVSALEYLHSRDVVYRDIKLENLMLDKDGHIKITDFGLCKEGISDGATMKTFCGTPEYLAPEVLEDNDYGRAVDWWGLGVVMYEMMCGRLPFYNQDHERLFELILMEEIRFPRTLSPEAKSLLAGLLKKDPKQRLGGGPSDAKEVMEHRFFLSINWQDVVQKKLLPPFKPQVTSEVDTRYFDDEFTAQSITITPPDRYDSLGLLELDQRTHFPQFSYSASIRE

SEQ ID NO: 10 - protein, 479 aa, Homo sapiens
MSDVTIVKEGWVQKRGEYIKNWRPRYFLLKTDGSFIGYKEKPQDVDLPYPLNNFSVAKCQLMKTERPKPNTFIIRCLQWTTVIERTFHVDTPEEREEWTEAIQAVADRLQRQEEERMNCSPTSQIDNIGEEEMDASTTHHKRKTMNDFDYLKLLGKGTFGKVILVREKASGKYYAMKILKKEVIIAKDEVAHTLTESRVLKNTRHPFLTSLKYSFQTKDRLCFVMEYVNGGELFFHLSRERVFSEDRTRFYGAEIVSALDYLHSGKIVYRDLKLENLMLDKDGHIKITDFGLCKEGITDAATMKTFCGTPEYLAPEVLEDNDYGRAVDWWGLGVVMYEMMCGRLPFYNQDHEKLFELILMEDIKFPRTLSSDAKSLLSGLLIKDPNKRLGGGPDDAKEIMRHSFFSGVNWQDVYDKKLVPPFKPQVTSETDTRYFDEEFTAQTITITPPEKYDEDGMDCMDNERRPHFPQFSYSASGRE

(19) United States
(12) Patent Application Publication
(10) Pub. No.: US 2025/0109444 A1
(43) Pub. Date: Apr. 3, 2025

(54) DNA HYPOMETHYLATION AS A PREDICTIVE MARKER FOR CANCER THERAPY

(71) Applicant: Fred Hutchison Cancer Center, Seattle, WA (US)

(72) Inventors: Michael Haffner, Seattle, WA (US); Pallabi Mustafi, Seattle, WA (US); Brian Hanratty, Seattle, WA (US)

(73) Assignee: Fred Hutchinson Cancer Center, Seattle, WA (US)

(21) Appl. No.: 18/905,924

(22) Filed: Oct. 3, 2024

Related U.S. Application Data
(60) Provisional application No. 63/587,674, filed on Oct. 3, 2023.

(51) Int. Cl. C12Q 1/6886; A61K 45/06

(52) U.S. Cl. CPC C12Q 1/6886; A61K 45/06; C12Q 2600/106; C12Q 2600/154

(57) ABSTRACT

DNA hypomethylation as a predictive marker for cancer therapy is described. The susceptibility of hypomethylated cancers to particular treatments, for example to AKT inhibitors and anthracyclines is described. The described susceptibilities can be used to direct enrollment of subjects into appropriate clinical trials and to guide treatment selection and administration based on global DNA methylation level.

### CROSS-REFERENCE TO RELATED APPLICATIONS

This application claims priority to U.S. Provisional Patent Application No. 63/587,674 filed Oct. 3, 2023, which is incorporated herein by reference in its entirety as if fully set forth herein.

### STATEMENT REGARDING FEDERALLY SPONSORED RESEARCH OR DEVELOPMENT

This invention was made with government support under W81XWH-21-1-0229 awarded by the Defense Health Agency, Medical Research and Development. The government has certain rights in the invention.

### REFERENCE TO SEQUENCE LISTING

The Sequence Listing associated with this application is provided in XML format in lieu of a paper copy and is hereby incorporated by reference into the specification. The name of the file containing the Sequence Listing is 3B94119.XML. The file is 11,036 bytes, was created on Oct. 2, 2024, and is being submitted electronically via Patent Center.

### FIELD OF THE DISCLOSURE

The present disclosure describes DNA hypomethylation as a predictive marker for cancer therapy. For example, the disclosure describes the susceptibility of DNA hypomethylated cancers to particular treatments, such as treatment with AKT inhibitors and/or anthracyclines. The described susceptibilities can be used to direct enrollment of subjects into appropriate clinical trials and to guide treatment selection and administration based on DNA methylation level.

### BACKGROUND OF THE DISCLOSURE

Cancer causes millions of deaths a year worldwide with rates rising as more people live to an older age. In 2024, it is estimated that over 2 million new cases of cancer will be diagnosed in the United States and over 600,000 people will die from the disease. Cancer is considered not one disease but several diseases and is considered a multifactorial disease, resulting from a combination of genetic and environmental factors, contributing to tumor heterogeneity.

Despite advances in cancer treatments, mortality associated with the disease remains too high. Thus, there remains a need in the art for more effective cancer therapies.

### SUMMARY OF THE DISCLOSURE

The present disclosure describes DNA hypomethylation as a predictive marker for cancer therapy. For example, the disclosure describes the susceptibility of DNA hypomethylated cancers (DHMC) to particular treatments, such as treatment with AKT inhibitors and/or anthracyclines. The described susceptibilities can be used to direct enrollment of subjects into appropriate clinical trials and to guide treatment selection and administration based on methylation level.

Particular embodiments include methods that identify subjects with global hypomethylation and recommend treatment with a DHMC-sensitive compound. Particular embodiments include method including obtaining a sample derived from a subject, determining a DNA methylation level of the sample, and determining that the subject would be sensitive to treatment with a DHMC-sensitive compound. In particular embodiments, a DHMC-sensitive compound includes an AKT inhibitor or an anthracycline. In particular embodiments, the methods further include enrolling a subject in a clinical trial. In particular embodiments, the methods further include administering the DHMC-sensitive compound to the subject, thereby treating the subject.

The present disclosure also shows that DHMC show sensitivity to DHMC-sensitive compounds irrespective of the status of AKT signaling intermediates (such as PTEN) and show increased sensitivity to DHMC-sensitive compounds when DNA hypomethylation is induced. In particular embodiments, hypomethylation is induced with a DNA methyltransferase (DNMT) inhibitor. In particular embodiments, methods include a secondary treatment including a DNMT inhibitor or a polycomb repressive complex 2 (PRC2) inhibitor.

The present disclosure also includes compositions and kits for treating a DHMC.

### BRIEF DESCRIPTION OF THE FIGURES

Some of the drawings submitted herewith may be better understood in color. Applicant considers the color versions of the drawings as part of the original submission and reserves the right to present color images of the drawings in later proceedings.

FIG. 1. Global methylation levels as determined by DNA methylation arrays in the TCGA pan-cancer atlas. DNA hypomethylation can be detected with variable degrees across all common tumor types.

FIGS. 2A, 2B. DNA hypomethylated tumors are highly sensitive to AKT inhibition in vitro. (2A) Cell viability curves for DHMC (black) and non-DHMC (light gray) cell lines treated with 3 different AKT inhibitors (afuresertib, AZD5363, ipatasertib). (2B) IC50s for DHMCs (black) and non-DHMCs (gray). Across a wide spectrum of cancer cell lines, DHMC show increased sensitivity compared to non-DHMCs to inhibitors of Akt.

FIGS. 3A, 3B. DNA hypomethylated associated AKTi sensitivity is independent of alterations in the AKT signaling pathway. (3A) Western blot shows expression and activity of AKT signaling intermediates. The effect on cell viability is independent on PTEN genomic and expression status. (3B) Cell viability curves for DHMC (square and inverted triangle) and non-DHMC (circle and triangle) show differences in cell viability to two AKTis. DHMC show increased sensitivity to AKTi irrespective of PTEN status.

FIGS. 4A, 4B. Modulation of global DNA methylation changes the sensitivity to AKT inhibitors. (4A) Cell viability curves for solvent (circle) and SAMe (square) treated LNCaP cells. The difference in cell viability in response to AKTi (ipatasertib) treatment is shown. (4B) Decitabine (DAC) and GSK344862 results in sensitization. Pharmacologically induced DNA hypomethylation, through treatment with DNA methyltransferase (DNMT) inhibitors decitabine and GSK-3484862, increases sensitivity to AKT inhibitors (AKTi). Conversely, restoring DNA methylation with prolonged treatment using the methyl donor S-Adenosylmethionine (SAMe) decreases sensitivity to AKTi.

FIGS. 5A-5C. AKTi synergizes with inhibition of the polycomb repressive complex 2 (PRC2) (5A) Western blot shows an increase in global H3K27me3 levels and a concomitant decrease in global histone levels following AKT inhibition (AKTi) in HT1376 and LNCaP cells. (5B) Co-treatment with the enhancer zeste homolog 2 (EZH2) inhibitor GSK126 and the AKT inhibitor ipatasertib results in a synergistic reduction in cell viability in LNCaP cells. (5C) Synergistic interaction is observed between the embryonic ectoderm development (EED) inhibitor A395 and ipatasertib in MALME3 cells. Increased dependency on PRC2-mediated chromatin repression is observed with AKT inhibition, creating a therapeutic vulnerability. Co-treatment with AKTi and PRC2 inhibitors (such as EZH2 or EED inhibitors) results in synergistic effects.

FIGS. 6A, 6B. DNA hypomethylated tumors are highly sensitive to anthracyclines with histone eviction activity. (6A) Cell viability curves for DHMC (black) and non-DHMC (gray) cell lines treated with histone evicting anthracyclines dimethyl-doxorubicin and aclarubicin. (6B) IC50s for DHMCs (black) and non-DHMCs (gray). Across a wide spectrum of cancer cell lines DHMC show increased sensitivity to histone evicting anthracycline drugs.

### DETAILED DESCRIPTION

Despite advances in cancer treatments, mortality associated with the disease remains too high. Thus, there remains a need in the art for more effective cancer therapies.

DNA methylation alterations are well recognized as drivers in cancer. Given the important role of DNA methylation in determining genome organization, gene expression and cell identity, it was reasoned that global loss of DNA methylation in cancers could identify cancer subtypes with distinct molecular features that could exhibit unique vulnerabilities that could be targeted therapeutically.

The present disclosure provides a new epigenetic subtype of cancer that is characterized by a significant loss of DNA methylation that sets this cancer type apart from others. Using both genetic and pharmacologic screens, it was found that DNA hypomethylated tumors are exquisitely sensitive to particular types of treatments, such as AKT inhibition and/or anthracyclines (e.g. aclarubicin).

The present disclosure describes the susceptibility of DNA hypomethylated cancers (DHMC) to treatment with DHMC-sensitive compounds, such as AKT inhibitors and/or anthracyclines. The susceptibilities described herein can be used to direct enrollment of subjects into appropriate clinical trials and to guide treatment selection and administration based on cancer methylation level.

Herein, a DHMC-sensitive compound is a molecule, protein, or chemical substance in which DHMC have increased sensitivity to. DHMC-sensitive compounds can be used to treat DHMC.

Particular embodiments include methods that identify subjects with global hypomethylation and recommend treatment with a DHMC-sensitive compound. Particular embodiments include method including obtaining a sample derived from a subject, determining a DNA methylation level of the sample, and determining that the subject would be sensitive to treatment with a DHMC-sensitive compound. In particular embodiments, a DHMC-sensitive compound includes an AKT inhibitor or an anthracycline. In particular embodiments, the methods further include enrolling a subject in a clinical trial. In particular embodiments, the methods further include administering the DHMC-sensitive compound to the subject, thereby treating the subject.

The present disclosure also shows that DHMC show sensitivity to DHMC-sensitive compounds irrespective of PTEN status and show increased sensitivity to DHMC-sensitive compounds when DNA hypomethylation is induced. In particular embodiments, hypomethylation is induced with a DNA methyltransferase (DNMT) inhibitor. In particular embodiments, methods include a secondary treatment including a DNMT inhibitor or a polycomb repressive complex 2 (PRC2) inhibitor.

The present disclosure also includes compositions and kits for treating a DHMC.

Aspects of the disclosure are now described in additional detail and with additional options to practice the disclosure as follows: (i) DNA Hypomethylated Cancers & Methylation Assays; (ii) AKT and AKT Inhibitors; (iii) Anthracyclines; (iv) Comparisons and Reference Levels; (v) Compositions for Administration; (vi) Kits; (vii) Treatments for DNA Hypomethylated Cancers; (viii) Exemplary Embodiments; (ix) Experimental Example; and (x) Closing Paragraphs. These headings are provided for organization purposes and do not limit the scope or interpretation of the disclosure.

### (i) DNA Hypomethylated Cancers & Methylation Assays

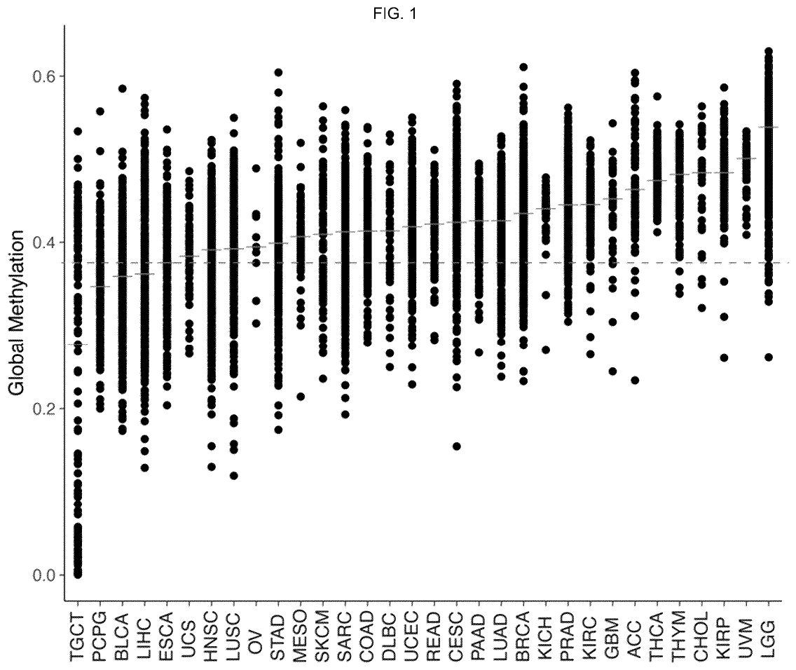

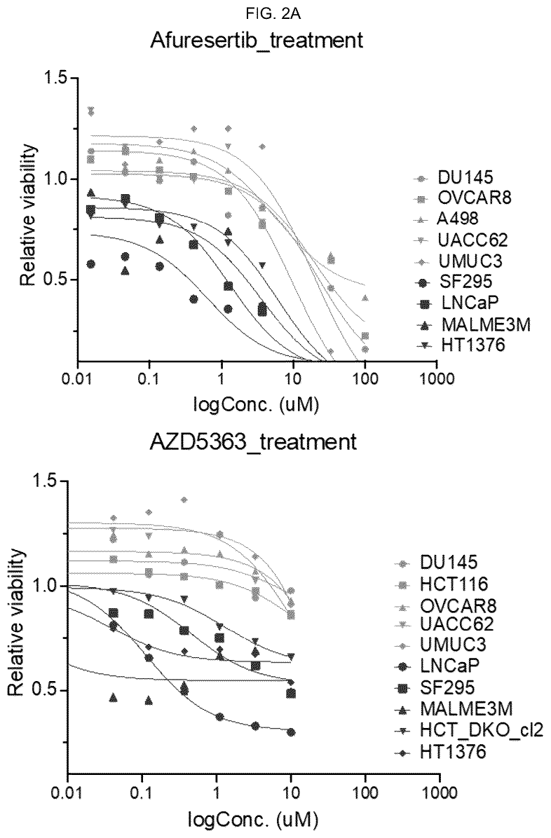

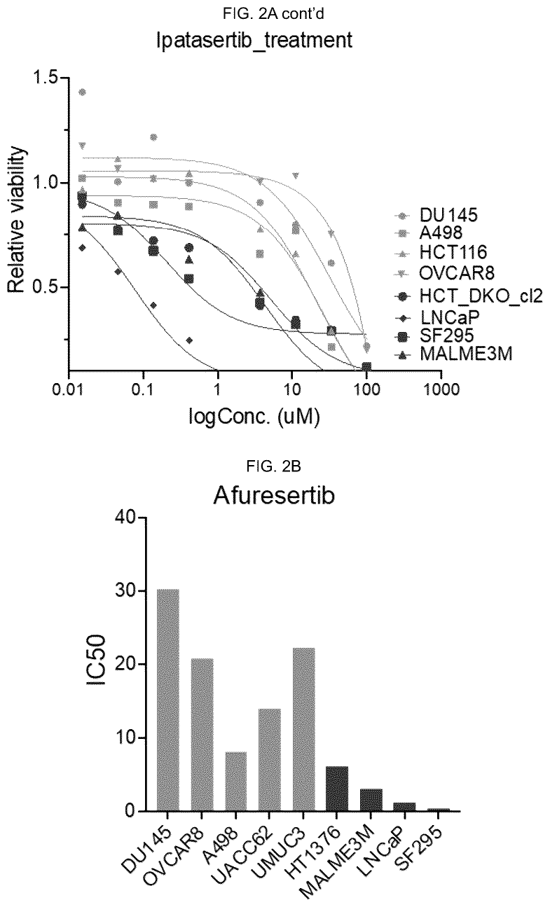

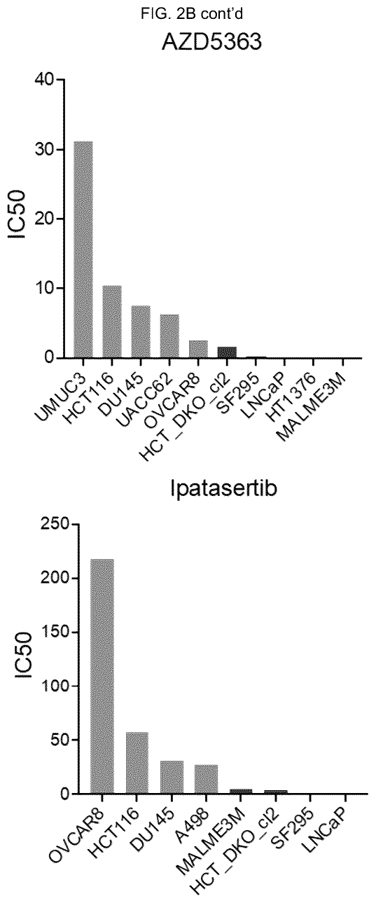

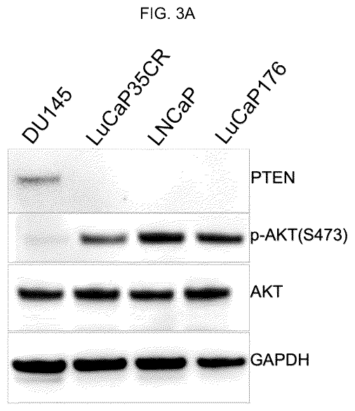

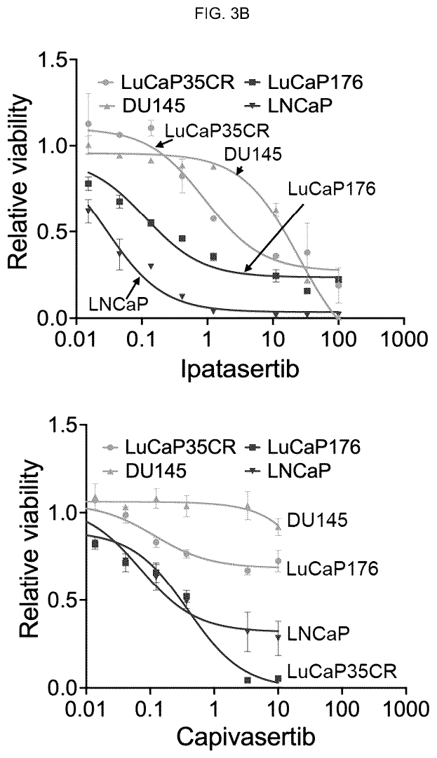

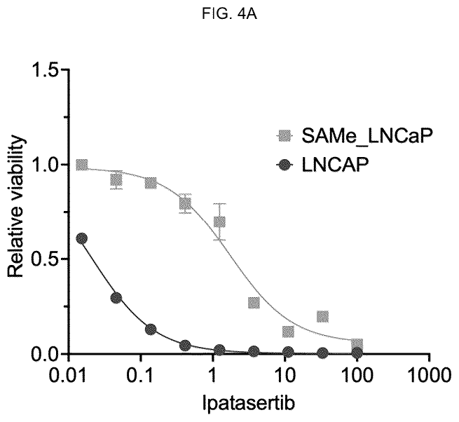

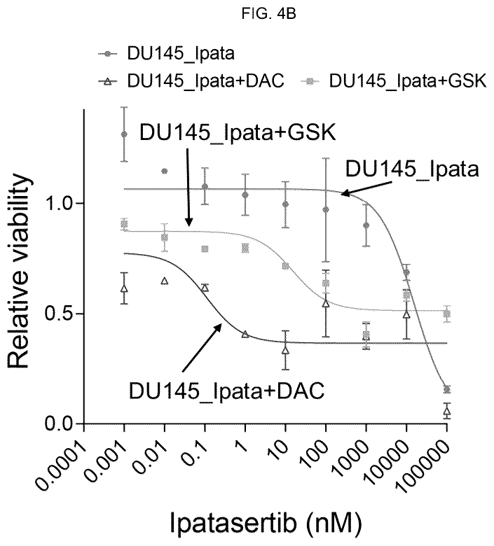

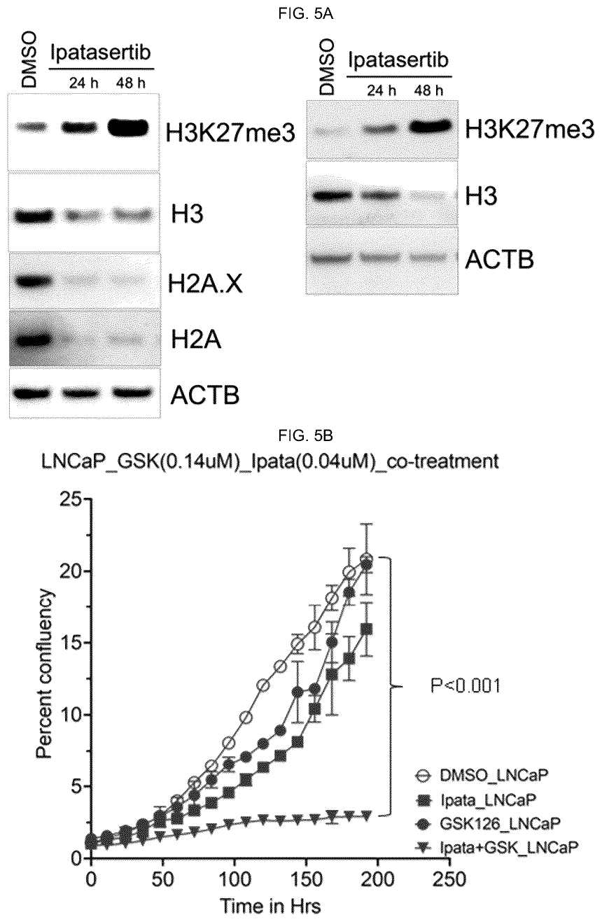

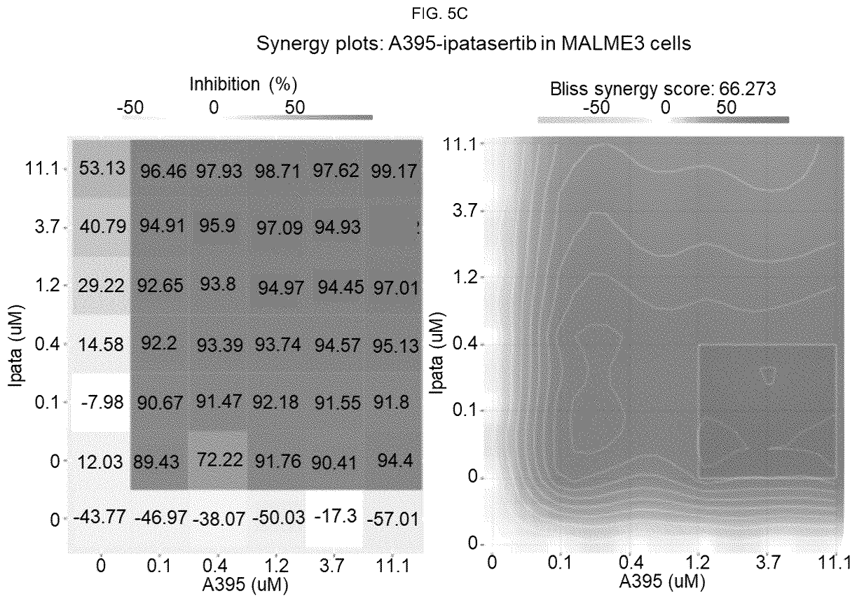

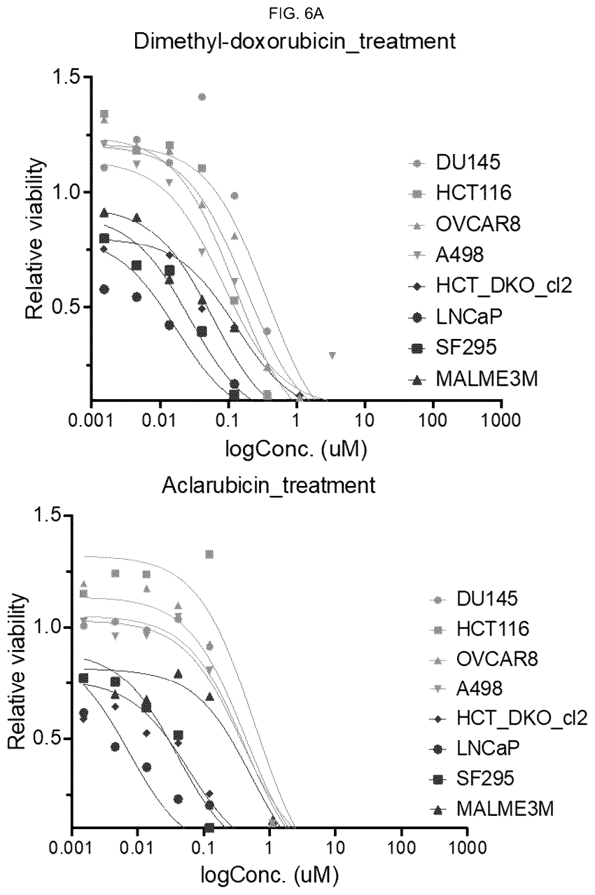

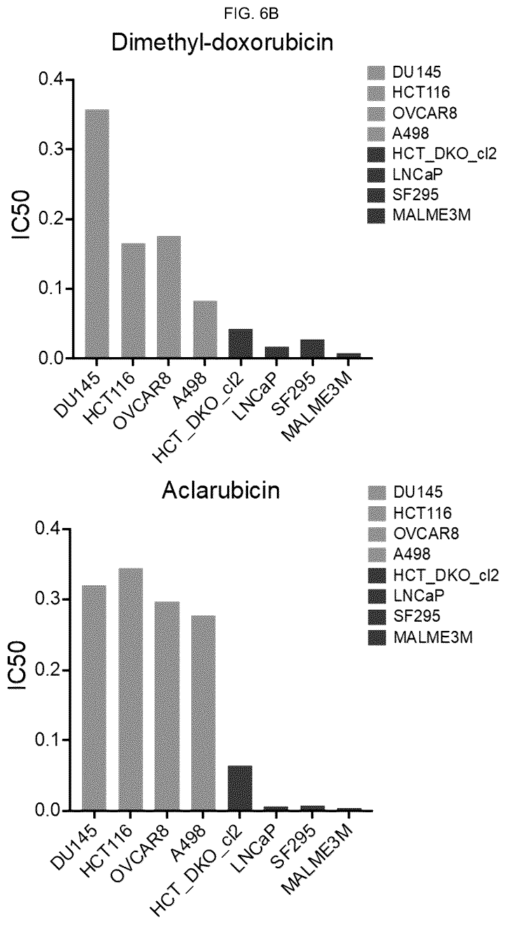

FIG. 1 provides global methylation levels as determined by DNA methylation arrays in the TCGA pan-cancer atlas. DNA hypomethylation can be detected in variable degrees across all common tumor types. Thus, one aspect of the disclosure includes obtaining a sample including DNA from a subject and determining the DNA methylation level. In particular embodiments, the sample includes a tissue biopsy sample or a liquid sample. A tissue biopsy is generally a mass of cells obtained from the subject's body by, for example, a surgeon, interventional radiologist, interventional cardiologist, or other specialized clinician. Often, the tissue biopsy is a tumor biopsy, meaning that the mass of cells is obtained from a primary, secondary, or metastatic tumor. In particular embodiments, the liquid sample includes blood, plasma, cerebrospinal fluid, sputum, stool, urine, lymphatic fluid, or saliva. In particular embodiments, the liquid sample includes cancer cells, cell free DNA (cfDNA), and/or circulating tumor DNA (ctDNA).

Methods described herein can be used to assess the DNA methylation level of a sample derived from a subject. In particular embodiments, the subject has cancer. In particular embodiments, the subject has a DNA hypomethylated cancer (DHMC). In particular embodiments, the cancer type is described in the TCGA pan-cancer atlas. In particular embodiments, the cancer includes an adrenal cancer, astrocytoma, bladder cancer, blood cancer, bone cancer, brain cancer, breast cancer, carcinoma, corpus uterine cancer, cervical cancer, colorectal cancer, colon cancer, chordoma, choroid plexus carcinoma, choroid plexus papilloma, ear, nose and throat (ENT) cancer, endometrial cancer, ependymoma, esophageal cancer, extragonadal germ cell tumor, gastrointestinal cancer, glioblastoma, head and neck cancer, hepatocellular carcinoma (e.g., HBV-induced), Hodgkin's disease, intestinal cancer, kidney cancer, larynx cancer, leukemia, liver cancer, lung cancer, lymph node cancer, lymphoma (e.g., diffuse large B-cell lymphoma, follicular lymphoma), leukemia, malignant rhabdoid tumor (e.g., atypical teratoid rhabdoid tumor, extrarenal rhabdoid tumor), medulloblastoma, melanoma, meningioma, mesothelioma, multiple myeloma, nasopharynx cancer, neuroblastoma, neuroglial tumor, non-Hodgkin's lymphoma, oligodendroglioma, oligoastrocytoma, oral cancer, ovarian cancer (e.g., ovarian clear cell adenocarcinoma, ovarian endometrioid adenocarcinoma, ovarian serous adenocarcinoma), pancreatic cancer (e.g., pancreatic ductal adenocarcinoma, pancreatic endocrine tumor), penile cancer, pharynx cancer, pineoblastoma, prostate cancer, rectal cancer, renal cell carcinoma, renal medullary carcinoma, sarcoma (e.g., clear cell sarcoma of soft tissue, carcinosarcoma, chondrosarcoma, Ewing sarcoma, gastrointestinal stromal tumor, osteosarcoma, rhabdomyosarcoma, epithelioid sarcoma, NOS sarcoma), seminoma, schwannoma, skin cancer, skin squamous cell carcinoma, stem cell cancer, stomach cancer, teratoma, testicular cancer, thyroid cancer, urothelial, uterine cancer, vaginal cancer, or vascular tumor for a methylation level.

Once an appropriate sample is obtained, the DNA methylation level of the sample can be assessed. In particular embodiments, the DNA methylation level includes the global DNA methylation level.

Methylation levels can be assessed using various methylation detection assays. A “methylation detection assay” refers to an assay, which can be commercially available, for distinguishing methylated versus unmethylated cytosine loci in DNA.

Particular embodiments assess methylation levels using immunolabeling. For example, Haffner et al. (Oncotarget. 2011, 2(8):627-37) describes immunolabeling to detect 5-methyl cytosine (5mC) and 5-hydroxymethylcytosine (5hmC). In these methods, positive controls for 5hmC staining optimization can be generated by transiently transfecting HEK293 cells with myc-tagged TET2 constructs or vector controls using Lipofectamine 2000 (Invitrogen, Carlsbad, CA). Cell pellets can be fixed in 10% buffered formalin and embedded in paraffin as described in Sfanos et al., PLoS One. 2011; 6:e20874. 5 micron paraffin sections can be de-waxed and rehydrated following standard protocols. Antigen retrieval can consist of steaming for 30 min in citrate buffer (pH 6.0) followed by incubation in 3.5 N HCl for 15 min at room temperature. Slides can be washed and equilibrated in TBS-Tween buffer (Sigma, St. Louis, MO) for 10 min. The same antigen retrieval can be used for 5mC and 5hmC. For immunolabeling of 5hmC, the rabbit polyclonal 5 hydroxymethylcytosine specific antibody (Active Motif, Cat #39769, Carlsbad, CA) can be applied at 1:20,000 dilution. For 5mC detection, the mouse monoclonal 5 methylcytosine specific antibody (Calbiochem, EMD Chemicals Inc., San Diego, CA) can be used at 1:2000 dilution. Both primary antibodies can be incubated for 1 h at room temperature. Immuno-complexes can be detected using the PowerVision+™ immunohistochemistry detection system from ImmunoVision Technologies Co (Norwell, MA, USA) with 3,3′-diaminobenzidine tetrahydrochloride (DAB) as the chromogen. After immunohistochemical staining, tissue sections can be counterstained with hematoxylin.

For immunofluorescence analysis, slides can be pretreated as outlined above and incubated with rabbit polyclonal 5hmC specific antibody (Active Motif, Cat #39769) at a 1:8000 dilution with or without mouse monoclonal antibodies specific to myc (9E11, Santa Cruz, CA), cytokeratin 34PE12-903 (ENZO, Farmingdale, NY) or cytokeratin 15 (Ab-1, NeoMarkers, Fremont, CA) at 1:50 dilutions. Immuno-complexes can be further labeled with secondary antibodies conjugated with Alexa 488 or Alexa 568 dyes (Invitrogen) and DNA can be counterstained with DAPI. Slides can then be visualized using a Nikon E400 fluorescence microscope (Nikon Instruments, Melville, NY). For additional information and methods regarding methylation detection through immunohistochemistry, see Haffner et al., Proc. Natl. Acad. Sci. USA. 2018, 115(37):e8580-e8582 Haffner et al., Proc. Natl. Acad. Sci. USA. 2018, 115(37):e8580-e8582; and Siska et al., Oncoimmunology. 2017, 6(4):e1305535.

The overall methylation level (as a percentage of total cytosine content) in genomic DNA can also be determined by a high-performance liquid chromatography/mass spectrometry method. In certain examples, 10 ng of genomic DNA can be resuspended in 50 μl of high-performance liquid chromatography grade water and digested with 4 units of nuclease P1 (Sigma) at 65° C. for 10 min in a digestion buffer containing 0.04 mM DFAM, 3.25 mM NH₄OAc, pH 5.0, 0.5 mM ZnCI₂ in a final volume of 100 μl. 20 μl of 100 mM Trizma base, pH 8.5, can be added, and this reaction can be treated with 4 units of alkaline phosphatase at 37° C. for 1 h. Following incubation, 20 μl of 300 mM NH₄OAc, pH 5.0, and 6 μl of 0.25 mM DFAM in 50 mM EDTA can be added. Quantitation of 5-methylcytosine and cytosine can be performed with an API 3000 LC/MS instrument (Applied Biosystems). Separation of free nucleotides formed after digestion and treatment with nuclease P1 and alkaline phosphatase can be performed on a 250×2.00 mm, 5-μm C18 column. 15 μl of samples or standards (consisting of serial dilutions of 5-methylcytosine and cytosine maintained in a buffer identical to samples) can be injected in triplicate along with 250 μl/min of a mobile phase profile consisting of 98% solution A (5 mM NH₄OAc, 0.1% formic acid, pH 3) and 2% solution B (90% acetonitrile) for 4 min, followed by a linear ramping to 60% solution A and 40% solution B in 1 min, then a ramping to 98% solution A and 2% solution B in 1 min, and finally maintaining this composition isocratically for the final 5 min. Cytosine can be monitored in MRM mode with the ion pair 227/112, while 5-methylcytosine was monitored in MRM mode with the ion pair 242/126. The quantity of each analyte can be calculated with reference to the standard dilution series, and the ratio of 5-methylcytosine to total cytosine (5-methylcytosine plus cytosine) can be calculated. For additional information regarding use of mass spectrometry to determine methylation level, see Agoston et al., Journal of Biological Chemistry, Vol. 280, Issue 18, P18302-18310 (2005) and Yegnasubramanian et al., Cancer Res. 2008, 68(21):8954-67.

Other methylation detection assays are known in the art and these methods can be used for absolute quantification or relative quantification of methylated nucleic acid. Such methylation assays include, among other techniques, two main steps. The first step is a methylation specific reaction or separation, such as (i) bisulfite treatment, (ii) enzymatic conversion (iii) methylation specific binding, or (iv) a methylation specific restriction enzyme. The second main step involves (i) amplification and detection, or (ii) direct detection by various methods, such as (a) PCR (sequence specific amplification), such as Taqman® (Roche Molecular Systems, Inc., Pleasanton, CA), (b) sequencing of bisulfite treated or enzymatically converted DNA, (c) sequencing by ligation of dye modified probes (including cycling ligation and cleavage), (d) pyrosequencing, (e) single molecule sequencing, (f) mass spectrometry, or (g) Southern blot analysis.

Techniques for measuring cytosine methylation also include methods resulting in methylation-based DNA sequence changes, such as bisulfite-based methylation assays and enzymatic methyl sequences to detect DNA methylation (EM-Seq).

The addition of bisulfite to DNA results in the methylation of unmethylated cytosine and its ultimate conversion to the nucleotide uracil. Uracil has similar binding properties to thiamine in the DNA sequence. Previously methylated cytosine does not undergo similar chemical conversion on exposure to bisulfite. Bisulfite assays can thus be used to discriminate previously methylated versus unmethylated cytosine.

An exemplary quantitative methylation detection assay combines bisulfite treatment and restriction analysis COBRA, which uses methylation sensitive restriction endonucleases, gel electrophoresis, and detection based on labeled hybridization probes. (Ziong and Laird, Nucleic Acid Res. 1997 25; 2532-4). Another exemplary detection assay is the methylation specific polymerase chain reaction PCR (MSPCR) for amplification of DNA segments of interest. This assay can be performed after sodium bisulfite conversion of cytosine and uses methylation sensitive probes. Other detection assays include the Quantitative Methylation (QM) assay, which combines PCR amplification with fluorescent probes designed to bind to putative methylation sites; MethyLight™ (Qiagen, Redwood City, CA) a quantitative methylation detection assay that uses fluorescence based PCR (Eads, et al., Cancer Res. 1999; 59:2302-2306); and Ms-SNuPE, a quantitative technique for determining differences in methylation levels in CpG sites. As with other techniques, Ms-SNuPE also requires bisulfite treatment to be performed first, leading to the conversion of unmethylated cytosine to uracil while methyl cytosine is unaffected. PCR primers specific for bisulfite converted DNA are then used to amplify sequences of interest. The amplified PCR product is isolated and used to quantitate the methylation level of CpG sites. (Gonzalgo and Jones Nuclei Acids Res 1997; 25:252-31). Probes can include other detectable labels besides fluorescent labels. For example, a detectable label can include a fluorescent label, dye, radioactive isotope, enzyme, magnetic bead, or biotin.

In particular embodiments, the INFINIUM® (Ilumina, Inc., San Diego California, USA) Human Methylation 450 Beadchip assay can be used. The Illumina assay can be used for genome wide quantitative methylation profiling. In particular embodiments, genomic DNA can be extracted from cells. Genomic DNA can be isolated and proteins or other contaminants can be removed from the DNA using proteinase K. The DNA can then be removed from the solution using available methods such as organic extraction, salting out, or binding the DNA to a solid phase support. As described above, and in the Infinium® Assay Methylation Protocol Guide, the DNA can be treated with sodium bisulfite. The bisulfite converted DNA can then be denatured and amplified. A next step can use enzymatic means to fragment the DNA. The fragmented DNA can then be precipitated using isopropanol and separated by centrifugation. The separated DNA can next be suspended in a hybridization buffer. The fragmented DNA can then be hybridized to beads that have been covalently limited to 50mer nucleotide segments at a locus specific to the cytosine nucleotide of interest in the genome. There are a total of over 500,000 bead types specifically designed to anneal to the locus where the particular cytosine is located, and the beads are bound to silicon-based arrays. There are two bead types designed for each locus, one bead type represents a probe that is designed to match to the methylated locus at which the cytosine nucleotide will remain unchanged. The other bead type corresponds to an initially unmethylated cytosine, which after sodium bisulfite treatment, is converted to uracil and ultimately a thiamine nucleotide. Unhybridized DNA (DNA not annealed to the beads) is washed away leaving only DNA segments bound to the appropriate bead and containing the cytosine of interest. If the cytosine of interest was unmethylated prior to the sodium bisulfite treatment, then it will match with the unmethylated or “U” bead probe. If the cytosine was methylated, single base mismatch will occur with the “U” bead probe oligomer. No further nucleotide extension on the bead oligomer occurs. This will lead to low fluorescent signal from the “U” bead. The reverse will happen on the “M” or methylated bead probe.

Lasers can then be used to stimulate fluorophores bound to the beads. The level of methylation at each cytosine locus is detected by the intensity of the fluorescence from the methylated compared to the unmethylated bead. Cytosine methylation level is expressed as “p” which is the ratio of the methylated-bead probe signal to total signal intensity at that cytosine locus.

Enzymatic methyl sequences to detect DNA methylation (EM-Seq) can also be used. Enzymatic detection of 5mC and 5hmC utilizes three enzymes and two reaction sets. Tet methylcytosine dioxygenase 2 (TET2) and T4-BGT protect 5mC and 5hmC from subsequent deamination by APOBEC3A. TET2 catalyzes the oxidization of 5mC to 5hmC, then 5-formylcytosine (5fC), and 5caC with the concomitant formation of CO₂ and succinate. T4-BGT catalyzes the glucosylation of both TET2-formed and genomic 5hmC to 5-(β-glucosyloxymethyl)cytosine (5gmC). APOBEC3A deaminates cytosines, but not the protected forms of 5mC or 5hmC, enabling their discrimination. For additional information regarding EM-Seq methods, see Vaisvila et al., Genome Res. 2021, 31(7):1280-1289.

In particular embodiments, pyrosequencing can be used to detect methylation level. Pyrosequencing is a method of DNA sequencing that relies on detection of the release of pyrophosphates as DNA is synthesized (and is therefore a “sequencing by synthesis” technique). To assess methylation by pyrosequencing, a DNA sample can be incubated with sodium bisulfite, converting unmethylated cytosine to uracil. The presence of uracil will result in thymine incorporation during PCR amplification. Therefore, sequencing results that include thymine at a nucleotide position that is known to encode cytosine can be interpreted as unmethylated sites. In contrast cytosines present in the sequencing results indicate that the site was methylated in the original DNA sample, because methylation protects cytosine from conversion to uracil upon treatment. Bisulfite treatment can also be performed on control samples with known methylation patterns, to reduce or eliminate false positive results. Commercially available pyrosequencing machines include Pyro Mark Q96 (Qiagen, Hilden, Germany). For more details on methods to use pyrosequencing for measurement of methylation, see Delaney et al., Methods Mol Biol. 2015 1343: 249-264. Pyrosequencing is especially useful for detecting methylation in the CpG sites within genes.

While not preferred, measurement of mRNA levels transcribed by genes with altered cytosine methylation can also be assessed to indirectly indicate methylation level. Any technique for determining expression levels of mRNA can be used including Northern blot analysis, fluorescent in situ hybridization (FISH), RNase protection assays (RPA), microarrays, PCR-based, or other technologies for measuring RNA levels can be used.

Up (hyper)- or down (hypo)-methylation also can be detected indirectly using, for example, cDNA arrays, cDNA fragment fingerprinting, cDNA sequencing, clone hybridization, differential display, differential screening, FRET detection, liquid microarrays, PCR, RT-PCR, quantitative RT-PCR analysis with TaqMan assays, molecular beacons, microelectric arrays, oligonucleotide arrays, polynucleotide arrays, serial analysis of gene expression (SAGE), and/or subtractive hybridization.

Further hybridization technologies that may be used are described in, for example, U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; and 5,800,992 as well as WO 95/21265; WO 96/31622; WO 97/10365; WO 97/27317; EP 373 203; and EP 785 280.

Various methylation detection assays use nucleic acids and/or proteins linked to chips, such as microarray chips. See, for example, U.S. Pat. Nos. 5,143,854; 6,087,112; 5,215,882; 5,707,807; 5,807,522; 5,958,342; 5,994,076; 6,004,755; 6,048,695; 6,060,240; 6,090,556; and 6,040,138. Binding to nucleic acids or proteins on microarrays can be detected by scanning the microarray with a variety of lasers or charge coupled device (CCD)-based scanners, and extracting features with software packages, for example, Imagene (Biodiscovery, Hawthorne, CA), Feature Extraction Software (Agilent), Scanalyze (Eisen, M. 1999. SCANALYZE User Manual; Stanford Univ., Stanford, Calif. Ver 2.32.), or GenePix (Axon Instruments). Embodiments disclosed herein can be used with high throughput screening (HTS).

Typically, HTS refers to a format that performs at least 100 assays, at least 500 assays, at least 1000 assays, at least 5000 assays, at least 10,000 assays, or more per day. When enumerating assays, either the number of samples or the number of markers assayed can be considered. Generally, HTS methods involve a logical or physical array of either samples, or the nucleic acid or protein markers, or both. Appropriate array formats include both liquid and solid phase arrays. For example, assays employing liquid phase arrays, e.g., for hybridization of nucleic acids, binding of antibodies or other receptors to ligand, etc., can be performed in multiwell or microtiter plates. Microtiter plates with 96, 384, or 1536 wells are widely available, and even higher numbers of wells, e.g., 3456 and 9600 can be used. In general, the choice of microtiter plates is determined by the methods and equipment, e.g., robotic handling and loading systems, used for sample preparation and analysis.

HTS assays and screening systems are commercially available from, for example, Zymark Corp. (Hopkinton, MA); Air Technical Industries (Mentor, OH); Beckman Instruments, Inc. (Fullerton, CA); Precision Systems, Inc. (Natick, MA), etc. These systems typically automate entire procedures including all sample and reagent pipetting, liquid dispensing, timed incubations, and final readings of the microplate in detector(s) appropriate for the assay. These configurable systems provide HTS as well as a high degree of flexibility and customization. The manufacturers of such systems provide detailed protocols for the various methods of HTS.

Methylation can be further assessed using various methylation detection methods such as: Tet-assisted pyridine borane sequencing (TAPS), plasma-based ctDNA assays, bead-based MBD fluorescence assays, plate-based (bead independent) MBD assays, bead-based MBD assays with a triplex probe mix, the iQuant™ NGS-HS dsDNA Assay Kit, single-nucleotide variant (SNV) assays, somatic copy number variant (CNV) assays, bisulfate conversion-based microarray assays, differential hybridization assays, methylated DNA immunoprecipitation based assays, methylated CpG island recovery assays, methylation sensitive high resolution melting assays, microarray assays, pyrosequencing assays, invasive cleavage amplification assays, sequencing by ligation based assays, mass spectrometry assays, restriction landmark genomic scanning, methylation-sensitive representational difference analysis (MS-RDA), bisulfate conversion, padlock probe hybridization, circularization, amplification, reduced representation bisulfite sequencing (RRBS), and next generation or multiplexed sequencing for high throughput detection of methylation.

Particular embodiments utilize ddPCR™ (Bio-Rad Laboratories, Hercules, CA). ddPCR technology uses a combination of microfluidics and surfactant chemistry to divide PCR samples into discrete partitions (i.e., water-in-oil droplets). Hindson et al., Anal. Chem. 83(22): 8604-8610 (2011). The droplets support PCR amplification of the target template molecules they contain and use reagents and workflows similar to those used for most standard Taqman probe-based assays. For example, within the context of the current disclosure, a representative droplet would include DNA for methylation analysis.

Amplification may be performed with any suitable reagents (e.g. template nucleic acid (e.g. DNA or RNA)), primers, probes, buffers, replication catalyzing enzymes (e.g. DNA polymerase, RNA polymerase), nucleotides, salts (e.g. MgCl₂), etc. In some embodiments, an amplification mixture includes any combination of at least one primer or primer pair, at least one probe, at least one replication enzyme (e.g., at least one polymerase), and deoxynucleotide (and/or nucleotide) triphosphates (dNTPs and/or NTPs), etc.

Following PCR, each droplet is analyzed or read in a flow cytometer to determine the fraction of PCR-positive droplets in the original sample. Detection can also be based on one or more characteristics of a sample partition such as a physical, chemical, luminescent, or electrical aspects, which correlate with amplification. These data are then analyzed using Poisson statistics to determine the target concentration in the original sample. See Bio-Rad Droplet Digital™ (ddPCR™) PCR Technology.

To determine whether DNA from a sample is hypomethylated, the methylation level of the DNA from the sample can be compared to the methylation level of DNA from a healthy tissue sample derived from the same subject or a subject without cancer. A sample is considered hypomethylated when the methylation level of the DNA within the sample is, for example, 10%, 20%, 30%, 40%, 50%, 60%, 70%, 80% or 90% of the level of the healthy tissue sample. In particular embodiments, global DNA methylation is assessed.

In particular embodiments, a sample is considered hypomethylated when the DNA methylation level of the sample results in a distinguishable difference from the DNA methylation level of a healthy sample. For example, a sample may be considered hypomethylated if the DNA from the sample is methylated in a range from 0% to 50%, 10% to 50%, 20% to 50%, or 30% to 50% as compared to the methylation of DNA from a healthy sample.

In particular embodiments, a sample is considered hypomethylated when a DNA molecule from the sample contains multiple CpG sites (e.g., more than 3, 4, 5, 6, 7, 8, 9, 10, etc.) and a high percentage of the CpG sites (e.g., any percentage within the range of 50%-100%) are unmethylated.

In particular embodiments, the methylation level is compared to a reference level as described elsewhere herein.

The methylation level can be transmitted within a report to an external device. A report may include one or more discrete items of information provided by a machine conducting an analysis (e.g., a data output) or provided by a medical professional via a suitable data entry interface. (e.g., a point-and-click interface). An initial report may include raw data or text and/or discrete information items provided by a machine. An intermediate report may be created, for instance, by augmenting an initial report with additional information or analysis. A final report may be created by incorporating requested changes or additions, and may be formatted according to one or more applicable formatting rules.

The external device to which a report is transmitted can be associated with a health care provider, such as a doctor or personnel involved in conducting a clinical trial. Associated with a health care provider means owns or used by a health care provider to receive information relevant to patient care. Examples of external devices that can be associated with a health care provider include cell phones, pagers, computer terminals, and database systems maintained by a health care provider's employer. In various implementations, a processor(s) is operably connected to one or more transceivers that transmit and/or receive data over one or more communication networks. For example, the transceiver(s) includes a network interface card (NIC), a network adapter, a local area network (LAN) adapter, or a physical, virtual, or logical address to connect to various external devices and/or systems. In various examples, the transceiver(s) includes any sort of wireless transceivers capable of engaging in wireless communication (e.g., radio frequency (RF) communication). For example, the communication network(s) includes one or more wireless networks that include a 3^{rd} Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) includes other wireless modems, such as a modem for engaging in WI-FI®, WIGIG®, WIMAX®, BLUETOOTH®, NFC, radio frequency identification (RFID), or infrared communication over the communication network(s).

The results of a subject's methylation level assessment can direct qualification to or disqualification from a clinical trial. A clinical trial is a carefully regimented research program that allows investigators to evaluate a new drug, medical device, or biologic (or a novel application of a known drug, medical device or biologic), in the treatment, prevention or diagnosis of a disease or condition. Specifically, a clinical trial allows for the determination of whether such a product is considered safe and effective, in light of the product's benefits relative to its risks. Additional eligibility requirements for inclusion in a clinical trial may be considered. For examples, the presence of specific biomarkers can be determined to further determine a treatment strategy or enrollment in a clinical trial.

There are many types of clinical trials, including: dose ranging studies, treatment trials, prevention trials, screening trials, and quality of life trials. Dose ranging studies test various doses of an agent, and compare which dosage works the best with the least side effects. In a treatment trial, a new treatment, a new combination of drugs, or a new approach to surgery or radiation therapy is evaluated for safety and efficacy. Prevention trials evaluate medicines, vitamins, vaccines, minerals or lifestyle changes in preventing the occurrence or recurrence of a disease. Screening trials test the best ways to detect certain diseases or health conditions, while quality of life trials explore ways to improve the comfort and quality of life for individuals with a disease or illness.

Typically, a clinical trial involves multiple stages, each concerned with a different aspect of testing the drug, device or biologic. Phase I studies are primarily designed to determine the effect of a new drug on a small population of healthy subjects. The drug is evaluated at different doses, and the rates and routes of absorption, metabolism and excretion are determined. Specifically, this phase is concerned with establishing the safety of a new drug, and may take several months to complete.

Drugs that pass through phase I testing for safety and efficacy evaluation in a larger population of individuals. These individuals (up to several hundred) are afflicted with the disease or condition for which the drug is being developed. These studies are often randomized (where one group of subjects will receive a placebo) and blinded (where the participants, and sometimes the investigators, are unaware whether they are receiving treatment or a placebo).

Phase Ill studies typically involve several thousand patients afflicted with the treated disease or illness. The results of a phase Ill study allow the F.D.A. or other regulatory body to determine whether the new drug offers any benefit or advantage over therapies currently on the market (i.e., evaluation of pharmacoeconomic considerations). It also helps to determine side effects or complications that may not have surfaced in smaller populations. These studies generally last for several years, and constitute the last phase before a sponsor may seek new drug approval from the Food and Drug Administration.

Even after F.D.A. approval, a phase IV study may be undertaken to support marketing claims, further study side effects or to explore various off label uses.

If the subject has a DHMC, the subject can qualify for and be enrolled in a clinical trial studying DHMC-sensitive compounds (e.g., AKT inhibitors and/or anthracyclines) for the treatment of the subject's cancer. In particular embodiments, the clinical trial is assessing the efficacy of a DHMC-sensitive compounds (e.g., AKT inhibitor and/or anthracycline) in the treatment of a urothelial cancer, a melanoma, a hepatocellular cancer, a lung cancer, a head and neck cancer, or a squamous cell carcinoma.

If the subject does not have a DHMC, the subject may be disqualified from and not enrolled in a clinical trial studying AKT inhibitors and/or anthracyclines for the treatment of the subject's cancer type. In particular embodiments, the clinical trial is assessing the efficacy of an AKT inhibitor and/or anthracycline in the treatment of a urothelial cancer, a melanoma, a hepatocellular cancer, a lung cancer, a head and neck cancer, or a squamous cell carcinoma.

### (ii) AKT and AKT Inhibitors

AKT (also known as protein kinase B (PKB) or related to A and C protein kinase (RAC-P)) is a serine threonine phosphorylation enzyme that has been shown to be involved in a diverse set of signaling pathways, including cellular survival and apoptosis, proliferation, differentiation, metabolism, protein synthesis, and stress responses. Three variants of the AKT family have been characterized: AKT-1, AKT-2, and AKT-3. AKT is located in the cytosol of unstimulated cells and translocates to the cell membrane following stimulation. In particular embodiments, a DHMC-sensitive compound includes an AKT inhibitor.

In particular embodiments, AKT-1 (Chain A [Homo sapiens]) includes the sequence: GAMDPRVTMNEFEYLKLLGKGTFGKVILVKEKATGRYYAMKILKKEVIVAKDEVAHTLTENRVL QNSRHPFLTALKYSFQTHDRLCFVMEYANGGELFFHLSRERVFSEDRARFYGAEIVSALDYLH SEKNVVYRDLKLENLMLDKDGHIKITDFGLCKEGIKDGATMKXFCGTPEYLAPEVLEDNDYGRA VDWWGLGVVMYEMMCGRLPFYNQDHEKLFELILMEEIRFPRTLGPEAKSLLSGLLKKDPKQRL GGGSEDAKEIMQHRFFAGIVWQHVYEKKLSPPFKPQVTSETDTRYFDEEFTAQMITITPPDQD DSMECVDSERRPHFPQFDYSASSTA (SEQ ID NO: 8).

In particular embodiments, AKT-2 [Homo sapiens] includes the sequence: MNEVSVIKEGWLHKRGEYIKTWRPRYFLLKSDGSFIGYKERPEAPDQTLPPLNNFSVAECQLM KTERPRPNTFVIRCLQWTTVIERTFHVDSPDEREEWMRAIQMVANSLKQRAPGEDPMDYKCG SPSDSSTTEEMEVAVSKARAKVTMNDFDYLKLLGKGTFGKVILVREKATGRYYAMKILRKEVIIA KDEVAHTVTESRVLQNTRHPFLTALKYAFQTHDRLCFVMEYANGGELFFHLSRERVFTEERAR FYGAEIVSALEYLHSRDVVYRDIKLENLMLDKDGHIKITDFGLCKEGISDGATMKTFCGTPEYLA PEVLEDNDYGRAVDWWGLGVVMYEMMCGRLPFYNQDHERLFELILMEEIRFPRTLSPEAKSLL AGLLKKDPKQRLGGGPSDAKEVMEHRFFLSINWQDVVQKKLLPPFKPQVTSEVDTRYFDDEFT AQSITITPPDRYDSLGLLELDQRTHFPQFSYSASIRE (SEQ ID NO: 9).

In particular embodiments, AKT-3 [Homo sapiens] includes the sequence: MSDVTIVKEGWVQKRGEYIKNWRPRYFLLKTDGSFIGYKEKPQDVDLPYPLNNFSVAKCQLMK TERPKPNTFIIRCLQWTTVIERTFHVDTPEEREEWTEAIQAVADRLQRQEEERMNCSPTSQIDNI GEEEMDASTTHHKRKTMNDFDYLKLLGKGTFGKVILVREKASGKYYAMKILKKEVIIAKDEVAHT LTESRVLKNTRHPFLTSLKYSFQTKDRLCFVMEYVNGGELFFHLSRERVFSEDRTRFYGAEIVS ALDYLHSGKIVYRDLKLENLMLDKDGHIKITDFGLCKEGITDAATMKTFCGTPEYLAPEVLEDND YGRAVDWWGLGVVMYEMMCGRLPFYNQDHEKLFELILMEDIKFPRTLSSDAKSLLSGLLIKDP NKRLGGGPDDAKEIMRHSFFSGVNWQDVYDKKLVPPFKPQVTSETDTRYFDEEFTAQTITITPP EKYDEDGMDCMDNERRPHFPQFSYSASGRE (SEQ ID NO: 10).

AKT promotes the phosphorylation of the molecules involved in apoptosis inhibition. AKT genes are amplified, or the protein is overexpressed, in a variety of cancers. Additionally, over-expression of active AKT often accompanies increased chemoresistance in cancer cells. Therefore, the AKT pathway is targeted to sensitize cancerous cells to one or more cancer treatment drugs. AKT activity is modulated by short peptides that are derivatives of the HJ loop of a serine/threonine kinase.

AKT activity may be inhibited by an AKT inhibitor such as acylated homoserine lactones; triciribine (TCN/API-2); triciribine phosphate (TCNP); 9-methoxy-2-methylellipticinium acetate; indazole-pyridine A-443654; isoform-specific allosteric kinase inhibitors; PI3K/AKT/mTOR inhibitors including: BEZ235, BKM120, Everolimus, MK-2206 dichloride, Pictilisib, LY294002, CAL-101, PI-3065, HS-173, PI-103, NU7441, TGX-221, 10-87114, Wortmannin, XL147, ZSTK474, BYL719, AS-605240, PIK-75, 3-methyladenine, A66, SAR245409, PIK-93, GSK2126458, PIK-90, PF-04691502, AZD6482, Apitolisib, GSK1059615, Duvelisib, Gedatolisib, TG100-1 15, AS-252424, BGT226, CUDC-907, PIK-294, AS-604850, GSK2636771, BAY80-6946, YM201636, CH5132799, CAY10505, rapamycin, and PIK-293; GSK690693, Ipatasertib (GDC-0068), Capivasertib (AZD5363), PF-04691502, AT7867, Triciribine (NSC 154020), CCT128930, A-674563, PHT-427, Miransertib (ARQ 092) HCl, Uprosertib (GSK2141795), Afuresertib (GSK2110183), AT13148, Miltefosine, Honokiol (NSC 293100), TIC10 Analogue, Deguelin, and TIC10 (ONC201).

AKT activity may be further inhibited by an AKT inhibitor such as derivatives of 3,4-dihydropyrazinol[2,3-b]pyranzine-2-[1H]-ones as discussed in patent publication WO2010062571 and antisense compounds including antisense oligonucleotides such as: RX-0194 including 5′ CCAGCCCCCACCAGTCCACT 3′ (SEQ ID NO: 1), RX-0201 including 5′ GCTGCATGATCTCCTTGGCG 3′(SEQ ID NO: 2), RX-0616 including 5′ AGATAGCTGGTGACAGACAG 3′(SEQ ID NO: 3), RX-0627 v5′ CGTGGAGAGATCATCTGAGG 3(SEQ ID NO: 4)′, RX-0628, including 5′ TCGAAAAGGTCAAGTGCTAC 3′(SEQ ID NO: 5), RX-0632, including 5′ TGGTGCAGCGGCAGCGGCAG 3′(SEQ ID NO: 6); RX-0638, including 5′ GGCGCGAGCGCGGGCCTAGC 3′(SEQ ID NO: 7); and other antisense compounds as discussed in U.S. Pat. Nos. 5,958,773 and 6,187,586.

### (iii) Anthracyclines

In particular embodiments, a DHMC-sensitive compound includes an anthracycline. Anthracyclines are drugs extracted from Streptomyces bacterium that are often used as antibiotics, antimicrobial agents, and in the treatment of various cancers. Anthracyclines have shown high anti-tumor activity and may be utilized as chemotherapeutics. Anthracyclines have a tetracyclic ring structure to which a sugar moiety is attached via a glycosidic linkage and anthracycline compounds act by intercalating with DNA. Anthracycline structures are based on the anthracyclinone (anthraquinone) or (naphthaquinone) core with quinones, phenolic groups, and other substituents on the resonating ring structure A, B, and C and on the saturated D ring (Formula (I)).

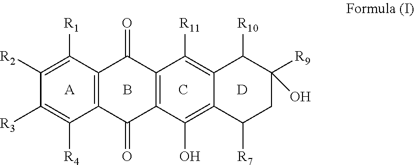

where R₁, R₂, R₃, R₄, R₇, R₉, R₁₀, R₁₁ may be an organic substituent, an inorganic substituent, an open-chain substituent, a branched substituent, a cyclic substituent, or any combination thereof. R₁, R₂, R₃, R₄, R₇, R₉, R₁₀, R₁₁ may be for example hydrogen, a hydroxyl group, a methoxy group, an acetyl group, a quinone, a phenolic group. In embodiments, water solubility may be provided to the anthracyclinone ring system by mono- or multisaccharide substitutions on the D ring system, most frequently at the R7 position.

Example formulas include:

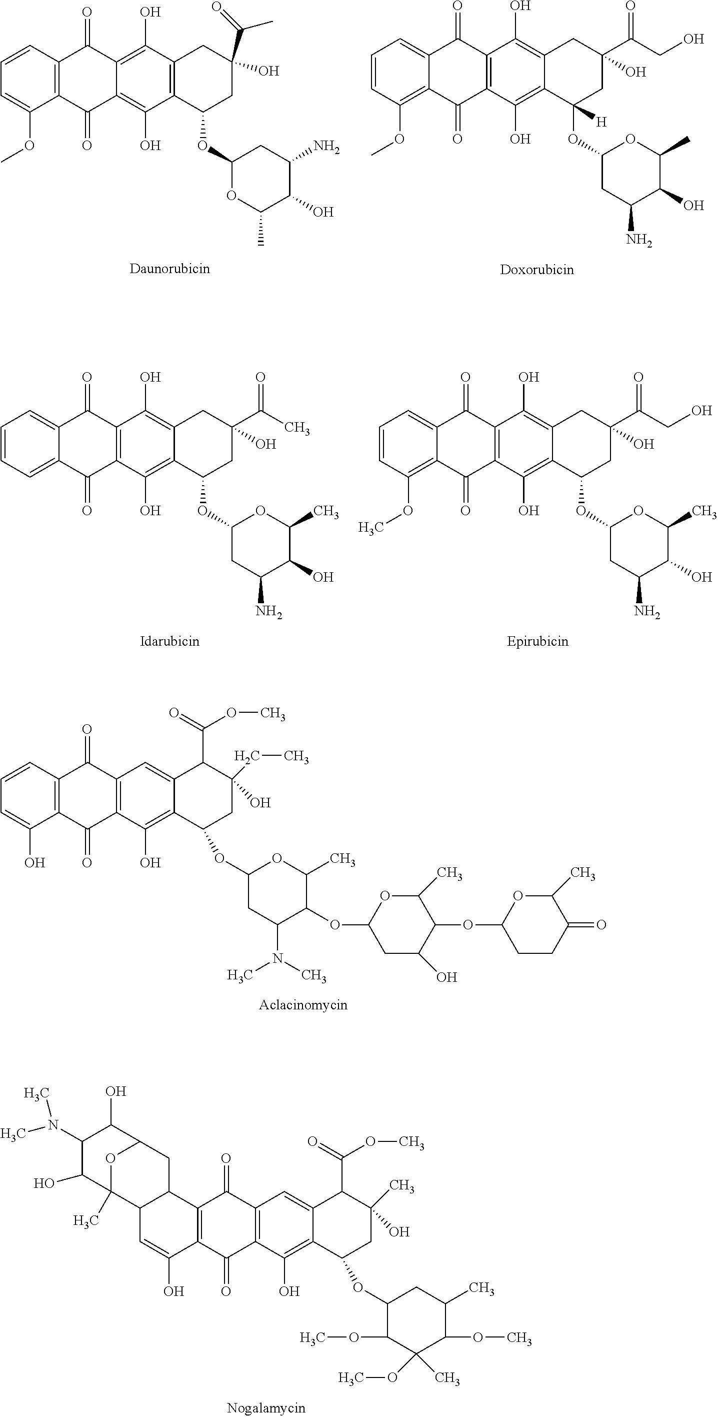

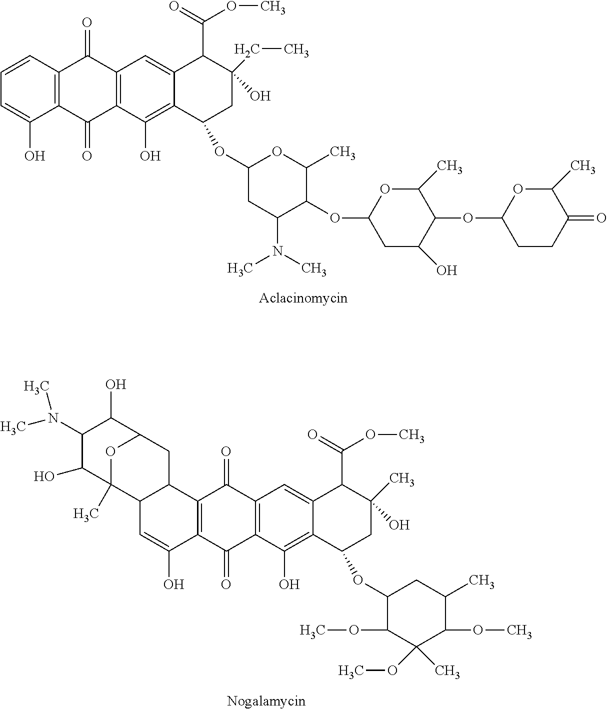

Without being bound by theory, functional characteristics of the anthracycline antibiotics, daunorubicin and doxorubicin, may include (1) the planar anthraquinone ring system, (2) quinone groups on the unsaturated rings, (3) stereochemistry of the D ring substitution at position 9, and (4) the amino sugar, daunosamine, which provides water solubility and chemical architecture for stabilizing DNA binding.

Additional examples of anthracycline compounds include: aclarubicin, aclacinomycin A, adriamycin, aklavin, AN-7A, AN-7B, AN-7D-apoprotein, auromomycin, carminomycin, cinerubin A, cinerubin B, cosmomycin A, dactinomycin, daunomycin, dimethyl-doxorubicin, ditrisarubicin A, ditrisarubicin B, ditrisarubicin C, 4′-epidoxorubicin, galirubin A, galirubin B, galirubin D, galirubin S, HBW-6(B), HBW-6(A), 4-demethoxydaunorubicin, MA 144-G1, MS 144-G2, MA 144-L, MA 144-M1, MA 144-M2, MA 144-N1, MA 144-S1, MA 144-S2, MA 144-U1, MA 144-U2, MA 144-Y, macromomycin, mitoxantrone (MTX), marcellomycin, musettamycin, NCS-apoprotein, nemorubicin, neocarzinostatin, pixantrone, plicamycin, pyrromycin, requinomycin, rhodomycin A, rhodomycin X, rhodomycin Y, rubidazone, rubidomycin, sabarubicin, steffimycin, trypanomycin, valrubicin, violamycin, and γ-rhodomycin Y. For more information and examples of anthracycline compounds see, for example: Bachur, Encyclopedia of Cancer (Second Edition), Academic Press, pages 57-61 (2002); Moini et al., Epidemiology of Endocrine Tumors, chapter 21, pages 473-488 (2020); U.S. Pat. Nos. 4,207,313; 4,303,785; 4,419,348; 4,612,371; 4,642,335; 5,744,454; 5,776,458; 9,674,673; 10,517,959; U.S. Pat. Pub. 2004/0202666; 2010/0074840; 2010/0092388; 2011/0171691; 2017/0360953; and 2019/0262464.

### (iv) Comparisons and Reference Levels

As indicated previously, up (hyper)- or down (hypo)-methylation of DNA (e.g., methylation status) can be assessed and a methylation level can be generated and compared to a relevant reference level. The methylation level can be one or more numerical values resulting from the assaying of a sample, and can be derived, e.g., by measuring methylation of the DNA in the sample by an assay, or from a dataset obtained from a provider such as a laboratory, or from a dataset stored on a server.

In the broadest sense, the methylation level may be qualitative or quantitative. As such, where detection is qualitative, the methods and kits provide a reading or evaluation, e.g., assessment, of whether or not the DNA is methylated in the sample being assayed. In further embodiments, the methods and kits provide a quantitative detection of methylation, i.e., an evaluation or assessment of the actual amount or relative abundance of DNA methylation in the sample being assayed. In such embodiments, the quantitative detection may be absolute or relative. As such, the term “quantifying” when used in the context of quantifying DNA methylation in a sample can refer to absolute or to relative quantification. Absolute quantification can be accomplished by inclusion of samples with known methylation parameters as one or more control samples and referencing, e.g., normalizing, the detected methylation level of the experimental sample with the known control sample (e.g., through generation of a standard curve). Alternatively, relative quantification can be accomplished by comparison of generated methylation level between two or more different samples to provide a relative quantification of each of the two or more samples, e.g., relative to each other. The actual measurement of methylation level can be determined using any method known in the art.

As stated previously, detected methylation levels can be compared to one or more reference levels. Reference levels can be obtained from one or more relevant datasets. A “dataset” as used herein is a set of numerical values resulting from evaluation of a sample (or population of samples) under a desired condition. The values of the dataset can be obtained, for example, by experimentally obtaining measures from sample(s) and constructing a dataset from these measurements. As is understood by one of ordinary skill in the art, the reference level can be based on e.g., any mathematical or statistical formula useful and known in the art for arriving at a meaningful aggregate reference level from a collection of individual datapoints; e.g., mean, median, median of the mean, etc. Alternatively, a reference level or dataset to create a reference level can be obtained from a service provider such as a laboratory, or from a database or a server on which the dataset has been stored.

A reference level from a dataset can be derived from previous measures derived from a population. A “population” is any grouping of subjects or samples of like specified characteristics. The grouping could be according to, for example, clinical parameters, clinical assessments, therapeutic regimens, or disease status. In particular embodiments, a population is a group of subjects without cancer. In particular embodiments, a population is a group of subjects with DHMC. In particular embodiments, a population is a group of subjects without DHMC.

In particular embodiments, hypomethylation is determined based on comparison to a reference level. The reference level can be based on the study of multiple control subjects, for example subjects having healthy tissue providing a reference level for comparison or subjects having a DHMC providing a reference level for comparison. When the reference level is derived from the study of multiple control subjects having healthy tissue, a DHMC would have a defined lower level of methylation than the reference level. When the reference level is derived from the study of multiple control subjects having a DHMC, a DHMC would be within a defined designated range of the reference level.

In particular embodiments, conclusions are drawn based on whether a methylation level is statistically significantly different or not statistically significantly different from a reference level. A measure is not statistically significantly different if the difference is within a level that would be expected to occur based on chance alone. In contrast, a statistically significant difference is one that is greater than what would be expected to occur by chance alone. Statistical significance or lack thereof can be determined by any of various methods well-known in the art. An example of a commonly used measure of statistical significance is the p-value. The p-value represents the probability of obtaining a given result equivalent to a particular datapoint, where the datapoint is the result of random chance alone. A result is often considered significant (not random chance) at a p-value less than 0.05.

In particular embodiments, obtained methylation levels can be subjected to an analytic process with chosen parameters. The parameters of the analytic process may be those disclosed herein or those derived using the guidelines described herein. The analytic process used to generate a result may be any type of process capable of identifying DHMCs based on methylation status detection, for example, a linear algorithm, a quadratic algorithm, a decision tree algorithm, or a voting algorithm. The analytic process may set a threshold for determining the probability that a sample belongs to a given class. The probability preferably is at least 60%, at least 70%, at least 80%, at least 90%, at least 95% or higher. Detection relies on performing a methylation assay on the biological sample.

The receiver operating characteristics (ROC) curve is a graph plotting sensitivity (true positive rate), which is defined in this setting as the percentage of hypomethylated cases with a positive test on the Y axis and false positive rate (1-specificity). False positive rate refers to the percentage of subjects without cancer falsely found to have a positive test.

The area under the ROC curves (AUC) indicates the accuracy of the test in identifying normal from hypomethylated cases (Hanley & McNeil, Radiology 1982; 143:29-36). The AUC is the area under the ROC plot from the curve to the diagonal line from the point of intersection of the X- and Y-axes and with an angle of incline of 45° (a test with no discrimination between two groups), has a 45° diagonal line from the lower left to the upper right corner). The higher the area under the receiver operating characteristics (ROC) curve, the greater the accuracy of the test in predicting the condition of interest. An area ROC=1.0 indicates a perfect test, which is positive in all cases with the disorder (e.g., HGD or EAC) and negative in all normal individuals without the disorder. Thus, the closer the plot is to the upper left corner, the higher the overall accuracy of the test.

### (v) Compositions for Administration

DHMC-sensitive compounds (active ingredients) can be formulated alone or in combination into compositions for administration to subjects. In particular embodiments, compositions include one active ingredient or, when relevant, a sequence encoding an active ingredient disclosed herein formulated with a pharmaceutically acceptable carrier. In particular embodiments, compositions include at least two active ingredients or sequences encoding active ingredients disclosed herein formulated with a pharmaceutically acceptable carrier.

In particular embodiments, compositions include an AKT inhibitor. In particular embodiments, compositions include an anthracycline. In particular embodiments, compositions include an AKT inhibitor and an anthracycline. When a composition includes an AKT inhibitor and an anthracycline, the AKT inhibitor and anthracycline can be included in the composition at a defined ration (e.g., AKT inhibitor:anthracycline at a ratio of 1:1, 1:2, 2:1).

Salts and/or pro-drugs of active ingredients can also be used. A pharmaceutically acceptable salt includes any salt that retains the activity of the active ingredients and is acceptable for pharmaceutical use. A pharmaceutically acceptable salt also refers to any salt which may form in vivo as a result of administration of an acid, another salt, or a prodrug which is converted into an acid or salt.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids.

Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine.

A prodrug includes an active ingredient which is converted to a therapeutically active compound after administration, such as by cleavage of an active ingredient or by hydrolysis of a biologically labile group.

In particular embodiments, the compositions include active ingredients of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA.

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the active ingredients or helps to prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

The compositions disclosed herein can be formulated for administration by, for example, injection, inhalation, infusion, perfusion, lavage, or ingestion. The compositions disclosed herein can further be formulated for intravenous, intradermal, intraarterial, intranodal, intralymphatic, intraperitoneal, intralesional, intraprostatic, intravaginal, intrarectal, topical, intrathecal, intratumoral, intramuscular, intravesicular, oral and/or subcutaneous administration and more particularly by intravenous, intradermal, intraarterial, intranodal, intralymphatic, intraperitoneal, intralesional, intraprostatic, intravaginal, intrarectal, intrathecal, intratumoral, intramuscular, intravesicular, and/or subcutaneous injection.

For injection, compositions can be formulated as aqueous solutions, such as in buffers including Hanks' solution, Ringer's solution, or physiological saline. The aqueous solutions can include formulatory agents such as suspending, stabilizing, and/or dispersing agents. Alternatively, the formulation can be in lyophilized and/or powder form for constitution with a suitable vehicle, e.g., sterile pyrogen-free water, before use.

For oral administration, the compositions can be formulated as tablets, pills, dragees, capsules, liquids, gels, syrups, slurries, suspensions and the like. For oral solid formulations such as powders, capsules and tablets, suitable excipients include binders (gum tragacanth, acacia, cornstarch, gelatin), fillers such as sugars, e.g. lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas. In the case of a pressurized aerosol, a dosage unit may be determined by providing a valve to deliver a metered amount. Capsules and cartridges of gelatin for use in an inhaler or insufflator may also be formulated including a powder mix of active ingredient and a suitable powder base such as lactose or starch.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salts.

Additionally, compositions can be formulated as sustained-release systems utilizing semipermeable matrices of solid polymers including at least one active ingredient. Various sustained-release materials have been established and are well known by those of ordinary skill in the art. Sustained-release systems may, depending on their chemical nature, release active ingredient following administration for a few weeks up to over 100 days. Depot preparations can be administered by injection; parenteral injection; instillation; or implantation into soft tissues, a body cavity, or occasionally into a blood vessel with injection through fine needles.

Depot formulations can include a variety of bioerodible polymers including poly(lactide), poly(glycolide), poly(caprolactone) and poly(lactide)-co(glycolide) (PLG) of desirable lactide:glycolide ratios, average molecular weights, polydispersities, and terminal group chemistries. Blending different polymer types in different ratios using various grades can result in characteristics that borrow from each of the contributing polymers.

The use of different solvents (for example, dichloromethane, chloroform, ethyl acetate, triacetin, N-methyl pyrrolidone, tetrahydrofuran, phenol, or combinations thereof) can alter microparticle size and structure in order to modulate release characteristics. Other useful solvents include water, ethanol, dimethyl sulfoxide (DMSO), N-methyl-2-pyrrolidone (NMP), acetone, methanol, isopropyl alcohol (IPA), ethyl benzoate, and benzyl benzoate.

Exemplary release modifiers can include surfactants, detergents, internal phase viscosity enhancers, complexing agents, surface active molecules, co-solvents, chelators, stabilizers, derivatives of cellulose, (hydroxypropyl)methyl cellulose (HPMC), HPMC acetate, cellulose acetate, pluronics (e.g., F68/F127), polysorbates, Span® (Croda Americas, Wilmington, Delaware), poly(vinyl alcohol) (PVA), Brij® (Croda Americas, Wilmington, Delaware), sucrose acetate isobutyrate (SAIB), salts, and buffers.

Excipients that partition into the external phase boundary of microparticles such as surfactants including polysorbates, dioctylsulfosuccinates, poloxamers, PVA, can also alter properties including particle stability and erosion rates, hydration and channel structure, interfacial transport, and kinetics in a favorable manner.

Additional processing of the disclosed sustained release depot formulations can utilize stabilizing excipients including mannitol, sucrose, trehalose, and glycine with other components such as polysorbates, PVAs, and dioctylsulfosuccinates in buffers such as Tris, citrate, or histidine. A freeze-dry cycle can also be used to produce very low moisture powders that reconstitute to similar size and performance characteristics of the original suspension.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by U.S. FDA Office of Biological Standards and/or other relevant foreign regulatory agencies.

In particular embodiments, compositions include therapeutic compositions. A therapeutic composition refers to a composition that treats a subject. A treatment can be detected by a reduction in a subject's disease or symptoms as described elsewhere herein.

### (vi) Kits

Also disclosed herein are kits for practicing methods disclosed herein. In particular embodiments, kits include one or more components to collect a sample from a subject, to process the sample and determine methylation level of DNA within the sample. In particular embodiments, kits include active ingredients or compositions including active ingredients.

Kits disclosed herein include a DHMC-sensitive compound such as an AKT inhibitor and/or an anthracycline.

Kits disclosed herein include materials to assay a sample for the methylation level as disclosed herein. In particular embodiments, the kits include materials to amplify sequences. In particular embodiments, the kits include materials to conduct PCR. Materials to conduct PCR include components of amplification mixtures, such as at least one primer or primer pair, at least one probe, at least one replication enzyme (e.g., at least one polymerase), and deoxynucleotide (and/or nucleotide) triphosphates (dNTPs and/or NTPs), etc. Particular embodiments provide the primer and probes sequences.

In particular embodiments, such kits include at least one polynucleotide that hybridizes to methylated sequences and at least one reagent for detecting gene methylation. Reagents for detecting methylation include, for example, sodium bisulfite, and/or methylation-sensitive or methylation-dependent restriction enzymes. In particular embodiments, the kit provides a solid support in the form of an assay device suitable for use in an assay.

Particular embodiments can include reference levels and/or control conditions (positive and/or negative).

In some embodiments, the kit include packaging material. As used herein, the term “packaging material” may refer to the physical structure that contains the components of the kit. In particular embodiments, the packaging material maintains sterility of the kit components and is made of materials commonly used for such purposes (e.g., paper, corrugated fiber, glass, plastic, foil, ampoule, etc.). Other materials useful for performing assays are included in the kit, including test tubes, pipettes, and the like.

Kits can include a notice prescribed by a governmental agency regulating the manufacture, use, or sale of diagnostics, pharmaceuticals or biological products, which notice reflects approval by the agency of manufacture, use, or sale for human use or administration.

Instructions for carrying out and interpreting methylation assays, including, optionally, instructions for generating a score, can also be included in a kit. Instructions can be provided in written, taped, videoed, VCR, CD-ROM, flashdrive, USB formats or can be provided on a website or other remote location.

In particular embodiments, kits exclude equipment (e.g., plate readers). In particular embodiments, kits exclude materials commonly found in laboratory settings (pipettes; test tubes; distilled H2O).

### (vii) Treatments for DNA Hypomethylated Cancers

Methods disclosed herein include treating subjects (humans, veterinary animals (dogs, cats, reptiles, birds, etc.) livestock (horses, cattle, goats, pigs, chickens, etc.) and research animals (monkeys, rats, mice, fish, etc.) with compositions disclosed herein. Treating subjects includes delivering therapeutically effective amounts. Therapeutically effective amounts include those that provide effective amounts, prophylactic treatments and/or therapeutic treatments.

An “effective amount” is the amount of a composition necessary to result in a desired physiological change in the subject. For example, an effective amount can provide an anti-cancer effect. Effective amounts are often administered for research purposes. Effective amounts disclosed herein can cause a statistically-significant effect in an animal model or in vitro assay relevant to the assessment of a cancer's development or progression.

A “prophylactic treatment” includes a treatment administered to a subject who does not display signs or symptoms of a cancer or displays only early signs or symptoms of a cancer such that treatment is administered for the purpose of diminishing or decreasing the risk of developing the cancer further. Thus, a prophylactic treatment functions as a preventative treatment against a cancer. In particular embodiments, prophylactic treatments reduce, delay, or prevent metastasis from a primary a cancer tumor site from occurring.

A “therapeutic treatment” includes a treatment administered to a subject who displays symptoms or signs of a cancer and is administered to the subject for the purpose of diminishing or eliminating those signs or symptoms of the cancer. The therapeutic treatment can reduce, control, or eliminate the presence or activity of the cancer and/or reduce control or eliminate side effects of the cancer.

Function as an effective amount, prophylactic treatment or therapeutic treatment are not mutually exclusive, and in particular embodiments, administered dosages may accomplish more than one treatment type.

In particular embodiments, therapeutically effective amounts provide anti-cancer effects. Anti-cancer effects include a decrease in the number of cancer cells, decrease in the number of metastases, a decrease in tumor volume, an increase in life expectancy, induced chemo- or radiosensitivity in cancer cells, inhibited angiogenesis near cancer cells, inhibited cancer cell proliferation, inhibited tumor growth, prevented or reduced metastases, prolonged subject life, reduced cancer-associated pain, and/or reduced relapse or re-occurrence of cancer following treatment.

A “tumor” is a swelling or lesion formed by an abnormal growth of cells (called neoplastic cells or tumor cells). A “tumor cell” is an abnormal cell that grows by a rapid, uncontrolled cellular proliferation and continues to grow after the stimuli that initiated the new growth cease. Tumors show partial or complete lack of structural organization and functional coordination with the normal tissue, and usually form a distinct mass of tissue, which may be benign, pre-malignant or malignant.

Compositions described herein can be utilized to treat any DHMC described in the TCGA pan-cancer atlas. The disclosed compositions can be used to treat hypomethylated adrenal cancers, hypomethylated astrocytomas, hypomethylated bladder cancers, hypomethylated blood cancers, hypomethylated bone cancers, hypomethylated brain cancers, hypomethylated breast cancers, hypomethylated carcinoma, hypomethylated cervical cancers, hypomethylated chordomas, hypomethylated choroid plexus carcinomas, hypomethylated choroid plexus papillomas, hypomethylated colon cancers, hypomethylated colorectal cancers, hypomethylated corpus uterine cancers, hypomethylated ear, nose and throat (ENT) cancers, hypomethylated endometrial cancers, hypomethylated ependymomas, hypomethylated esophageal cancers, hypomethylated extragonadal germ cell tumors, hypomethylated gastrointestinal cancers, hypomethylated glioblastomas, hypomethylated head and neck cancers, hypomethylated hepatocellular carinomas, hypomethylated Hodgkin's disease, hypomethylated intestinal cancers, hypomethylated kidney cancers, hypomethylated larynx cancers, hypomethylated leukemias, hypomethylated liver cancers, hypomethylated lung cancers, hypomethylated lymph node cancers, hypomethylated lymphomas, hypomethylated lung cancers, hypomethylated malignant rhabdoid tumors, hypomethylated medulloblastomas, hypomethylated melanomas, hypomethylated meningiomas, hypomethylated mesothelioma, hypomethylated multiple myelomas, hypomethylated myelomas, hypomethylated nasopharynx cancers, hypomethylated neuroblastomas, hypomethylated neuroglial tumors, hypomethylated non-Hodgkin's lymphoma, hypomethylated oligoastrocytomas, hypomethylated oligodendrogliomas, hypomethylated oral cancers, hypomethylated ovarian cancers, hypomethylated pancreatic cancers, hypomethylated penile cancers, hypomethylated pharynx cancers, hypomethylated pineoblastomas, hypomethylated prostate cancers, hypomethylated rectal cancers, hypomethylated renal cell carcinomas, hypomethylated renal medullary carcinomas, hypomethylated sarcomas, hypomethylated seminomas, hypomethylated schwannomas, hypomethylated skin cancers, hypomethylated skin squamous cell carcinomas, hypomethylated stem cell cancers, hypomethylated stomach cancers, hypomethylated teratomas, hypomethylated testicular cancers, hypomethylated thyroid cancers, hypomethylated urothelial cancers, hypomethylated uterine cancers, hypomethylated vaginal cancers, hypomethylated vascular tumors, and hypomethylated metastases thereof.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of cancer, stage of cancer, previous or concurrent therapeutic interventions, idiopathy of the subject and route of administration.

Useful doses can range from 0.1 to 5 μg/kg or from 0.5 to 1 μg/kg. In other examples, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly).

In particular embodiments, doses may be administered concurrently or sequentially within a clinically relevant time period. In particular embodiments, at least two active ingredients may be administered concurrently or sequentially within a clinically relevant time period.

The pharmaceutical compositions described herein can be administered by, for example, injection, inhalation, infusion, perfusion, lavage or ingestion. Routes of administration can include intravenous, intradermal, intraarterial, intraparenteral, intranasal, intranodal, intralymphatic, intraperitoneal, intralesional, intraprostatic, intravaginal, intrarectal, topical, intrathecal, intratumoral, intramuscular, intravesicular, oral, subcutaneous, and/or sublingual administration and more particularly by intravenous, intradermal, intraarterial, intraparenteral, intranasal, intranodal, intralymphatic, intraperitoneal, intralesional, intraprostatic, intravaginal, intrarectal, topical, intrathecal, intratumoral, intramuscular, intravesicular, oral, subcutaneous, and/or sublingual injection.

In particular embodiments, a patient can be monitored for changes in methylation level. When a global hypomethylation level is observed, treatments according to the current disclosure can be administered.

In certain embodiments, compositions are administered to a patient in conjunction with (e.g., before, simultaneously or following) any number of relevant treatment modalities. In particular embodiments, relevant treatment modalities can include chemotherapy, surgery, administration of immunosuppressive agents, administration of immunoablative agents, or irradiation. In particular embodiments, relevant treatment modalities can include administration of DNA methyltransferase (DNMT) inhibitors, Polycomb Repressive Complex 2 (PRC2) inhibitors, chemotherapeutic agents, anti-inflammatory agents, and/or cytokines.

In particular embodiments, pharmacologically induced DNA hypomethylation (e.g., by administration of a DNA methyltransferase (DNMT) inhibitor increases sensitivity to an Akt inhibitor. In particular embodiments, compositions disclosed herein may be administered in conjunction with any number of DNMT inhibitors. Examples of DNMT inhibitors include decitabine, GSK-3484862, azacytidine, hydralazine, procaine, MG98, or zebularine. In particular embodiments, an Akt inhibitor or anthracycline may be administered in conjunction with decitabine. In particular embodiments, an Akt inhibitor or anthracycline may be administered in conjunction with GSK-3484862.

Polycomb Repressive Complex 2 (PRC2)-mediated chromatin repression is observed with AKT inhibition (AKTi), creating a therapeutic vulnerability. In particular embodiments, compositions disclosed herein (e.g., Akt inhibitor or anthracycline) may be administered in conjunction with any number of PRC2 inhibitors. Examples of PRC2 inhibitors include enhancer zeste homolog 2 (EZH2) inhibitors or embryonic ectoderm development (EED) inhibitors. In particular embodiments, an EZH2 inhibitor includes GSK126, MAK683, EPZ6438, EPZ005687, CPI-1205, PF-06821497, SHR2554, UNC1999, valemetostat tosylate, CPI-169, UNC6852, or A-395. In particular embodiments, an EED inhibitor includes EED226, ORIC-944, LG1980, MAK683, A-395, BR-001, EEDi-5285, EEDi-5273, FTX-6058, EED162, EED709, or Jarid2114-118-K116 me3. In particular embodiments, an Akt inhibitor or anthracycline may be administered in conjunction with EZH2. In particular embodiments, an Akt inhibitor or anthracycline may be administered in conjunction with an EED inhibitor.

In certain embodiments, compositions disclosed herein may be administered in conjunction with any number of chemotherapeutic agents. Examples of chemotherapeutic agents include alkylating agents; aziridines; ethylenimines and methylamelamines; nitrogen mustards; nitrosureas; antibiotics; anti-metabolites); folic acid analogues; purine analogs; pyrimidine analogs; androgens; anti-adrenals; folic acid replenishers; platinum analogs; topoisomerase inhibitors; retinoic acid derivatives; and pharmaceutically acceptable salts, acids or derivatives of any of the above. Also included in this definition are anti-hormonal agents that act to regulate or inhibit hormone action on tumors such as anti-estrogens and anti-androgens; and pharmaceutically acceptable salts, acids or derivatives of any of the above. Combinations of chemotherapeutic agents are also administered where appropriate, including, CHOP, i.e., Cyclophosphamide (Cytoxan®), Doxorubicin (hydroxydoxorubicin), Vincristine (Oncovin®), and Prednisone.

In additional embodiments, the compositions disclosed herein can be administered with an anti-inflammatory agent. Anti-inflammatory agents or drugs include steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Exemplary NSAIDs include ibuprofen, naproxen, naproxen sodium, Cox-2 inhibitors, and sialylates. Exemplary analgesics include acetaminophen, oxycodone, tramadol of proporxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofm) and intramuscular) and minocycline.

In certain embodiments, the compositions described herein are administered in conjunction with a cytokine. “Cytokine” as used herein is meant to refer to proteins released by one cell population that act on another cell as intercellular mediators. Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGFα and TGFβ; insulin-like growth factor-I and -II; erythropoietin (EPO); osteoinductive factors; interferons such as interferon-alpha, beta, and - gamma; colony stimulating factors (CSFs) such as macrophage-CSF (M-CSF); granulocyte-macrophage-CSF (GM-CSF); and granulocyte-CSF (G-CSF); interleukins (IIs) such as IL-1, IL-1 alpha, IL-2, IL-3, IL-4, IL-5, IL-6, IL-7, IL-8, IL-9, IL-10, IL-II, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

In some embodiments, the secondary treatment is administered at the same time or within one week after the administration of the engineered cell or nucleic acid. In other embodiments, the secondary treatment is administered from 1 to 4 weeks or from 1 week to 1 month, 1 week to 2 months, 1 week to 3 months, 1 week to 6 months, 1 week to 9 months, or 1 week to 12 months after the administration of the first treatment (e.g., AKT inhibitor or anthracycline). In other embodiments, the secondary treatment is administered at least 1 month before administering the first treatment (e.g., AKT inhibitor or anthracycline). In some embodiments, the methods further include administering two or more secondary treatment.

The Exemplary Embodiments and Experimental Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (viii) Exemplary Embodiments

1. A method including:
identifying a subject having DNA hypomethylated cancer (DHMC); and administering to the subject a therapeutically effective amount of a DHMC-sensitive compound.

2. The method of embodiment 1, wherein the DHMC cancer includes an adrenal cancer, astrocytoma, bladder cancer, breast cancer, colon cancer, chordoma, choroid plexus carcinoma, choroid plexus papilloma, endometrial cancer, ependymoma, extragonadal germ cell tumor, glioblastoma, head and neck cancer, hepatocellular carcinoma, intestinal cancer, kidney cancer, leukemia, lung cancer, lymphoma, leukemia, malignant rhabdoid tumor, medulloblastoma, melanoma, meningioma, mesothelioma, multiple myeloma, neuroglial tumor, pancreatic cancer, oligodendroglioma, oligoastrocytoma, ovarian cancer, pineoblastoma, prostate cancer, renal cell carcinoma, renal medullary carcinoma, sarcoma, stem cell cancer, stomach cancer, testicular cancer, urothelial cancer, or uterine cancer.

3. The method of embodiments 1 or 2, wherein the DHMC cancer includes a urothelial cancer, a melanoma, a hepatocellular cancer, a lung cancer, a head and neck cancer, or a squamous cell carcinoma.

4. The method of any of embodiments 1-3, further including assessing a subject's eligibility for a clinical trial and recommending enrollment of the subject in the clinical trial.

5. The method of any of embodiments 1-4, wherein the DHMC-sensitive compound includes an AKT inhibitor or an anthracycline.

6. The method of embodiment 5, wherein the AKT inhibitor includes an acylated homoserine lactone, triciribine (TCN/API-2), triciribine phosphate (TCNP), 9-methoxy-2-methylellipticinium acetate, indazole-pyridine A-443654, an isoform-specific allosteric kinase inhibitor, or a PI3K/AKT/mTOR inhibitor.

7. The method of embodiments 5 or 6, wherein the AKT inhibitor includes BEZ235, BKM120, Everolimus, MK-2206 dichloride, Pictilisib, LY294002, CAL-101, PI-3065, HS-173, PI-103, NU7441, TGX-221, 10-87114, Wortmannin, XL147, ZSTK474, BYL719, AS-605240, PIK-75, 3-methyladenine, A66, SAR245409, PIK-93, GSK2126458, PIK-90, PF-04691502, AZD6482, Apitolisib, GSK1059615, Duvelisib, Gedatolisib, TG100-1 15, AS-252424, BGT226, CUDC-907, PIK-294, AS-604850, GSK2636771, BAY80-6946, YM201636, CH5132799, CAY10505, rapamycin, PIK-293, GSK690693, Ipatasertib (GDC-0068), Capivasertib (AZD5363), PF-04691502, AT7867, Triciribine (NSC 154020), CCT128930, A-674563, PHT-427, Miransertib (ARQ 092) HCl, Uprosertib (GSK2141795), Afuresertib (GSK2110183), AT13148, Miltefosine, Honokiol (NSC 293100), TIC10 Analogue, Deguelin, or TIC10 (ONC201).

8. The method of any of embodiments 5-7, wherein the AKT inhibitor includes an antisense oligonucleotide.

9. The method of embodiment 8, wherein the antisense oligonucleotide includes a sequence as set forth in SEQ ID NO: 1, SEQ ID NO: 2, SEQ ID NO: 3, SEQ ID NO: 4, SEQ ID NO: 5, SEQ ID NO: 6, or SEQ ID NO: 7 or a sequence having at least 95% sequence identity to the sequence as set forth in SEQ ID NO: 1, SEQ ID NO: 2, SEQ ID NO: 3, SEQ ID NO: 4, SEQ ID NO: 5, SEQ ID NO: 6, or SEQ ID NO: 7.

10. The method of any of embodiments 5-9, wherein the anthracycline includes a compound of Formula I:

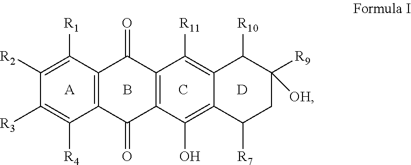

wherein:
R₁, R₂, R₃, R₄, R₇, R₉, R₁₀, R₁₁ are each independently selected in each instance from a group consisting of an organic substituent, an inorganic substituent, an open-chain substituent, a branched substituent, a cyclic substituent, or any combination thereof.

11. The method of embodiment 10, wherein the R₁, R₂, R₃, R₄, R₇, R₉, R₁₀, R₁₁ are each independently selected in each instance from a group consisting of a hydrogen, a hydroxyl group, a methoxy group, an acetyl group, a quinone, or a phenolic group.

12. The method of embodiments 10 or 11, wherein R₇ includes a mono- or multisaccharide substitution.

13. The method of any of embodiments 5-12, wherein the anthracycline includes aclarubicin, aclacinomycin A, adriamycin, aklavin, AN-7A, AN-7B, AN-7D-apoprotein, auromomycin, carminomycin, cinerubin A, cinerubin B, cosmomycin A, dactinomycin, daunomycin, daunorubicin, dimethyl-doxorubicin, ditrisarubicin A, ditrisarubicin B, ditrisarubicin C, doxorubicin, epirubicin, galirubin A, galirubin B, galirubin D, galirubin S, HBW-6(B), HBW-6(A), idarubicin, MA 144-G1, MS 144-G2, MA 144-L, MA 144-M1, MA 144-M2, MA 144-N1, MA 144-S1, MA 144-S2, MA 144-U1, MA 144-U2, MA 144-Y, macromomycin, marcellomycin, mitoxantrone, musettamycin, NCS-apoprotein, nemorubicin, neocarzinostatin, nogalamycin, pixantrone, plicamycin, pyrromycin, requinomycin, rhodomycin A, rhodomycin X, rhodomycin Y, rubidazone, rubidomycin, sabarubicin, steffimycin, trypanomycin, valrubicin, violamycin, or γ-rhodomycin Y.

14. The method of any of embodiments 1-13, wherein the administering includes injection, infusion, perfusion, lavage, or ingestion.

15. The method of any of embodiments 1-14, further including administering a secondary treatment.

16. The method of embodiment 15, wherein the secondary treatment includes administering a DNA methyltransferase (DNMT) inhibitor.

17. The method of embodiment 16, wherein the DNMT inhibitor includes decitabine, GSK-3484862, azacytidine, hydralazine, procaine, MG98, or zebularine.

18. The method of any of embodiments 15-17, wherein the secondary treatment includes administering a polycomb repressive complex 2 (PRC2) inhibitor.

19. The method of embodiment 18, wherein the PRC2 inhibitor includes enhancer zeste homolog 2 (EZH2) inhibitor or an embryonic ectoderm development (EED) inhibitor.

20. The method of embodiment 19, wherein the EZH2 inhibitor includes GSK126, MAK683, EPZ6438, EPZ005687, CPI-1205, PF-06821497, SHR2554, UNC1999, valemetostat tosylate, CPI-169, UNC6852, or A-395.

21. The method of embodiments 19 or 20, wherein the EED inhibitor includes EED226, ORIC-944, LG1980, MAK683, A-395, BR-001, EEDi-5285, EEDi-5273, FTX-6058, EED162, EED709, or Jarid2114-118-K116 me3.

22. The method of any of embodiments 15-21, wherein the secondary treatment includes chemotherapy, radiation, or surgery.

23. The method of any of embodiments 15-22, wherein the secondary treatment is administered simultaneously with the DHMC-sensitive compound.

24. The method of any of embodiments 15-22, wherein secondary treatment is administered from 1 to 4 weeks or from 1 week to 1 month, 1 week to 2 months, 1 week to 3 months, 1 week to 6 months, 1 week to 9 months, or 1 week to 12 months after administering the DHMC-sensitive compound.

25. A method including:
obtaining a sample derived from a subject;
processing the sample to determine a DNA methylation level of the sample; and
determining that the methylation level is hypomethylated and that the subject will be sensitive to treatment with a DNA hypomethylate cancer (DHMC)-sensitive compound.

26. The method of embodiment 25, wherein the DHMC-sensitive compound includes an AKT inhibitor and/or an anthracycline.

27. The method of embodiments 25 or 26, wherein the subject has cancer.

28. The method of embodiment 27, wherein the cancer includes an adrenal cancer, astrocytoma, bladder cancer, breast cancer, colon cancer, chordoma, choroid plexus carcinoma, choroid plexus papilloma, endometrial cancer, ependymoma, extragonadal germ cell tumor, glioblastoma, head and neck cancer, hepatocellular carcinoma, intestinal cancer, kidney cancer, leukemia, lung cancer, lymphoma, leukemia, malignant rhabdoid tumor, medulloblastoma, melanoma, meningioma, mesothelioma, multiple myeloma, neuroglial tumor, pancreatic cancer, oligodendroglioma, oligoastrocytoma, ovarian cancer, pineoblastoma, prostate cancer, renal cell carcinoma, renal medullo carcinoma, sarcoma, stem cell cancer, stomach cancer, testicular cancer, urothelial cancer, or uterine cancer.

29. The method of embodiments 27 or 28, wherein the cancer includes a urothelial cancer, a melanoma, a hepatocellular cancer, a lung cancer, a head and neck cancer, or a squamous cell carcinoma.

30. The method of any of embodiments 25-29, wherein the sample includes a tissue biopsy sample.

31. The method of embodiment 30, wherein the tissue biopsy sample includes a tumor biopsy sample.

32. The method of any of embodiments 25-29, wherein the sample includes a liquid sample.

33. The method of embodiment 32, wherein the liquid sample includes blood, plasma, cerebrospinal fluid, sputum, stool, urine, lymphatic fluid, or saliva.

34. The method of embodiments 32 or 33, wherein the liquid sample includes circulating tumor DNA (ctDNA).

35. The method of any of embodiments 25-34, wherein the methylation level is a global methylation level.

36. The method of any of embodiments 25-35, wherein the processing the sample includes extracting DNA from the sample.

37. The method of any of embodiments 25-36, wherein the processing the sample includes performing an immunostaining assay, mass spectrometry, or Enzymatic Methyl-seq (EM-Seq).

38. The method of any of embodiments 25-37, wherein the processing the sample includes: contacting the sample with an antibody that binds to 5-methyl cytosine (5mC), wherein the antibody is labeled with a detectable label;
detecting a signal representing a presence of the detectable label; and
determining the methylation level based on a signal intensity.

39. The method of any of embodiments 25-38, wherein the processing the sample includes: treating extracted DNA with bisulfite;
amplifying the bisulfite-treated DNA;
sequencing the amplified DNA to create sequence data; and
analyzing the sequence data, thereby determining the methylation level.

40. The method of embodiment 39, wherein the amplifying includes performing a polymerase chain reaction (PCR).

41. The method of any of embodiments 25-40, wherein the processing the sample further includes partitioning bisulfite treated DNA into partitions.

42. The method of embodiment 41, wherein each partition includes an amplification mixture.

43. The method of embodiment 42, wherein the amplification mixture includes at least one primer or primer pair, at least one probe, at least one replication enzyme, and deoxynucleotide and/or nucleotide triphosphates.

44. The method of any of embodiments 25-43, further including generating a report including data indicative of the methylation level.

45. The method of any of embodiments 25-44, further including assessing a subject's eligibility for enrollment in a clinical trial.

46. The method of any of embodiments 25-45, further including recommending enrollment of the subject in a clinical trial.

47. The method of embodiment 46, wherein the clinical trial assesses use of the DHMC-sensitive compound to treat cancer.

48. The method of any of embodiments 25-47, further including treating the subject with the DHMC-sensitive compound.

49. The method of any of embodiments 44-48, wherein the report provides a recommendation to administer a recommended treatment to the subject.

50. The method of embodiment 49, wherein the recommended treatment includes administering the DHMC-sensitive compound.

51. The method of any of embodiments 44-50, further including transmitting the report to an external device.

52. The method of embodiment 51, wherein the external device is associated with a health care provider.

53. The method of any of embodiments 26-52, wherein the AKT inhibitor includes an acylated homoserine lactone, triciribine (TCN/API-2), triciribine phosphate (TCNP), 9-methoxy-2-methylellipticinium acetate, indazole-pyridine A-443654, an isoform-specific allosteric kinase inhibitor, or a PI3K/AKT/mTOR inhibitor.

54. The method of any of embodiments 26-53, wherein the AKT inhibitor includes BEZ235, BKM120, Everolimus, MK-2206 dichloride, Pictilisib, LY294002, CAL-101, PI-3065, HS-173, PI-103, NU7441, TGX-221, 10-87114, Wortmannin, XL147, ZSTK474, BYL719, AS-605240, PIK-75, 3-methyladenine, A66, SAR245409, PIK-93, GSK2126458, PIK-90, PF-04691502, AZD6482, Apitolisib, GSK1059615, Duvelisib, Gedatolisib, TG100-1 15, AS-252424, BGT226, CUDC-907, PIK-294, AS-604850, GSK2636771, BAY80-6946, YM201636, CH5132799, CAY10505, rapamycin, PIK-293, GSK690693, Ipatasertib (GDC-0068), Capivasertib (AZD5363), PF-04691502, AT7867, Triciribine (NSC 154020), CCT128930, A-674563, PHT-427, Miransertib (ARQ 092) HCl, Uprosertib (GSK2141795), Afuresertib (GSK2110183), AT13148, Miltefosine, Honokiol (NSC 293100), TIC10 Analogue, Deguelin, or TIC10 (ONC201).

55. The method of any of embodiments 26-54, wherein the AKT inhibitor includes an antisense oligonucleotide.

56. The method of embodiment 55, wherein the antisense oligonucleotide has a sequence as set forth in SEQ ID NO: 1, SEQ ID NO: 2, SEQ ID NO: 3, SEQ ID NO: 4, SEQ ID NO: 5, SEQ ID NO: 6, or SEQ ID NO: 7 or a sequence having at least 95% sequence identity to the sequence as set forth in SEQ ID NO: 1, SEQ ID NO: 2, SEQ ID NO: 3, SEQ ID NO: 4, SEQ ID NO: 5, SEQ ID NO: 6, or SEQ ID NO: 7.

57. The method of any of embodiments 26-56, wherein the anthracycline includes a compound of Formula I:

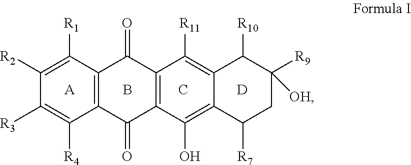

wherein:
R₁, R₂, R₃, R₄, R₇, R₉, R₁₀, R₁₁ are each independently selected in each instance from a group consisting of an organic substituent, an inorganic substituent, an open-chain substituent, a branched substituent, a cyclic substituent, or any combination thereof.

58. The method of embodiment 57, wherein the R₁, R₂, R₃, R₄, R₇, R₉, R₁₀, R₁₁ are each independently selected in each instance from a group consisting of a hydrogen, a hydroxyl group, a methoxy group, an acetyl group, a quinone, or a phenolic group.

59. The method of embodiments 57 or 58, wherein R₇ includes a mono- or multisaccharide substitution.

60. The method of any of embodiments 26-59, wherein the anthracycline includes aclarubicin, aclacinomycin A, adriamycin, aklavin, AN-7A, AN-7B, AN-7D-apoprotein, auromomycin, carminomycin, cinerubin A, cinerubin B, cosmomycin A, dactinomycin, daunomycin, daunorubicin, dimethyl-doxorubicin, ditrisarubicin A, ditrisarubicin B, ditrisarubicin C, doxorubicin, epirubicin, galirubin A, galirubin B, galirubin D, galirubin S, HBW-6(B), HBW-6(A), idarubicin, MA 144-G1, MS 144-G2, MA 144-L, MA 144-M1, MA 144-M2, MA 144-N1, MA 144-S1, MA 144-S2, MA 144-U1, MA 144-U2, MA 144-Y, macromomycin, marcellomycin, mitoxantrone, musettamycin, NCS-apoprotein, nemorubicin, neocarzinostatin, nogalamycin, pixantrone, plicamycin, pyrromycin, requinomycin, rhodomycin A, rhodomycin X, rhodomycin Y, rubidazone, rubidomycin, sabarubicin, steffimycin, trypanomycin, valrubicin, violamycin, or γ-rhodomycin Y.

61. The method of any of embodiments 49-60, wherein the administering includes injection, infusion, perfusion, lavage, or ingestion.

62. The method of any of embodiments 25-61, further including administering a secondary treatment.

63. The method of embodiment 62, wherein the secondary treatment includes administering a DNA methyltransferase (DNMT) inhibitor.

64. The method of embodiment 63, wherein the DNMT inhibitor includes decitabine, GSK-3484862, azacytidine, hydralazine, procaine, MG98, or zebularine.

65. The method of any of embodiments 62-64, wherein the secondary treatment includes administering a polycomb repressive complex 2 (PRC2) inhibitor.

66. The method of embodiment 65, wherein the PRC2 inhibitor includes enhancer zeste homolog 2 (EZH2) inhibitor or an embryonic ectoderm development (EED) inhibitor.

67. The method of embodiment 66, wherein the EZH2 inhibitor includes GSK126, MAK683, EPZ6438, EPZ005687, CPI-1205, PF-06821497, SHR2554, UNC1999, valemetostat tosylate, CPI-169, UNC6852, or A-395.

68. The method of embodiments 66 or 67, wherein the EED inhibitor includes EED226, ORIC-944, LG1980, MAK683, A-395, BR-001, EEDi-5285, EEDi-5273, FTX-6058, EED162, EED709, or Jarid2114-118-K116 me3.

69. The method of any of embodiments 62-68, wherein the secondary treatment includes chemotherapy, radiation, or surgery.

70. The method of any of embodiments 62-69, wherein the secondary treatment is administered simultaneously with the DHMC-sensitive compound.

71. The method of any of embodiments 62-69, wherein secondary treatment is administered from 1 to 4 weeks or from 1 week to 1 month, 1 week to 2 months, 1 week to 3 months, 1 week to 6 months, 1 week to 9 months, or 1 week to 12 months after administering the DHMC-sensitive compound.

72. A composition including i) a therapeutically effective amount of a DNA hypomethylated cancer (DHMC)-sensitive compound and ii) a pharmaceutically acceptable carrier, wherein the therapeutically effective amount treats DHMC.

73. The composition of embodiment 72, wherein the DHMC-sensitive compound includes an AKT inhibitor or an anthracycline.

74. The composition of embodiment 72, wherein the composition includes an AKT inhibitor and an anthracycline.

75. The composition of any of embodiments 73-74, wherein the AKT inhibitor includes an acylated homoserine lactone, triciribine (TCN/API-2), triciribine phosphate (TCNP), 9-methoxy-2-methylellipticinium acetate, indazole-pyridine A-443654, an isoform-specific allosteric kinase inhibitor, or a PI3K/AKT/mTOR inhibitor.

76. The composition of any of embodiments 73-75, wherein the AKT inhibitor includes BEZ235, BKM120, Everolimus, MK-2206 dichloride, Pictilisib, LY294002, CAL-101, PI-3065, HS-173, PI-103, NU7441, TGX-221, 10-87114, Wortmannin, XL147, ZSTK474, BYL719, AS-605240, PIK-75, 3-methyladenine, A66, SAR245409, PIK-93, GSK2126458, PIK-90, PF-04691502, AZD6482, Apitolisib, GSK1059615, Duvelisib, Gedatolisib, TG100-1 15, AS-252424, BGT226, CUDC-907, PIK-294, AS-604850, GSK2636771, BAY80-6946, YM201636, CH5132799, CAY10505, rapamycin, PIK-293, GSK690693, Ipatasertib (GDC-0068), Capivasertib (AZD5363), PF-04691502, AT7867, Triciribine (NSC 154020), CCT128930, A-674563, PHT-427, Miransertib (ARQ 092) HCl, Uprosertib (GSK2141795), Afuresertib (GSK2110183), AT13148, Miltefosine, Honokiol (NSC 293100), TIC10 Analogue, Deguelin, or TIC10 (ONC201).

77. The composition of any of embodiments 73-76, wherein the AKT inhibitor includes an antisense oligonucleotide.

78. The composition of embodiment 77, wherein the antisense oligonucleotide has a sequence as set forth in SEQ ID NO: 1, SEQ ID NO: 2, SEQ ID NO: 3, SEQ ID NO: 4, SEQ ID NO: 5, SEQ ID NO: 6, or SEQ ID NO: 7 or a sequence having at least 95% sequence identity to the sequence as set forth in SEQ ID NO: 1, SEQ ID NO: 2, SEQ ID NO: 3, SEQ ID NO: 4, SEQ ID NO: 5, SEQ ID NO: 6, or SEQ ID NO: 7.

79. The composition of any of embodiments 73-78, wherein the anthracycline includes a compound of Formula I:

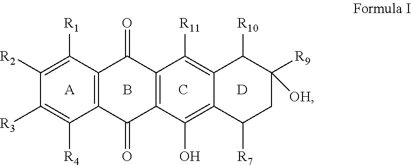

wherein:
R₁, R₂, R₃, R₄, R₇, R₉, R₁₀, R₁₁ are each independently selected in each instance from a group consisting of an organic substituent, an inorganic substituent, an open-chain substituent, a branched substituent, a cyclic substituent, or any combination thereof.

80. The composition of embodiment 79, wherein the R₁, R₂, R₃, R₄, R₇, R₉, R₁₀, R₁₁ are each independently selected in each instance from a group consisting of a hydrogen, a hydroxyl group, a methoxy group, an acetyl group, a quinone, or a phenolic group.

81. The composition of embodiments 79 or 80, wherein R₇ includes a mono- or multisaccharide substitution.

82. The composition of any of embodiments 73-81, wherein the anthracycline includes aclarubicin, aclacinomycin A, adriamycin, aklavin, AN-7A, AN-7B, AN-7D-apoprotein, auromomycin, carminomycin, cinerubin A, cinerubin B, cosmomycin A, dactinomycin, daunomycin, daunorubicin, dimethyl-doxorubicin, ditrisarubicin A, ditrisarubicin B, ditrisarubicin C, doxorubicin, epirubicin, galirubin A, galirubin B, galirubin D, galirubin S, HBW-6(B), HBW-6(A), idarubicin, MA 144-G1, MS 144-G2, MA 144-L, MA 144-M1, MA 144-M2, MA 144-N1, MA 144-S1, MA 144-S2, MA 144-U1, MA 144-U2, MA 144-Y, macromomycin, marcellomycin, mitoxantrone, musettamycin, NCS-apoprotein, nemorubicin, neocarzinostatin, nogalamycin, pixantrone, plicamycin, pyrromycin, requinomycin, rhodomycin A, rhodomycin X, rhodomycin Y, rubidazone, rubidomycin, sabarubicin, steffimycin, trypanomycin, valrubicin, violamycin, or γ-rhodomycin Y.

83. The composition of any of embodiments 72-82, further including a DNA methyltransferase (DNMT) inhibitor.

84. The composition of embodiment 83, wherein the DNMT inhibitor includes decitabine, GSK-3484862, azacytidine, hydralazine, procaine, MG98, or zebularine.

85. The composition of any of embodiments 72-84, further including a polycomb repressive complex 2 (PRC2) inhibitor.

86. The composition of embodiment 85, wherein the PRC2 inhibitor includes enhancer zeste homolog 2 (EZH2) inhibitor or an embryonic ectoderm development (EED) inhibitor.

87. The composition of embodiment 86, wherein the EZH2 inhibitor includes GSK126, MAK683, EPZ6438, EPZ005687, CPI-1205, PF-06821497, SHR2554, UNC1999, valemetostat tosylate, CPI-169, UNC6852, or A-395.

88. The composition of embodiments 86 or 87, wherein the EED inhibitor includes EED226, ORIC-944, LG1980, MAK683, A-395, BR-001, EEDi-5285, EEDi-5273, FTX-6058, EED162, EED709, or Jarid2114-118-K116 me3.

89. The composition of any of embodiments 72-88, further including a chemotherapeutic agent.

90. A kit including a DNA hypomethylated cancer (DHMC)-sensitive compound and reagents to detect methylation status of DNA.

91. The kit of embodiment 90, wherein the DHMC-sensitive compound includes an AKT inhibitor and/or an anthracycline.

92. The kit of embodiment 91, wherein the AKT inhibitor includes an acylated homoserine lactone, triciribine (TCN/API-2), triciribine phosphate (TCNP), 9-methoxy-2-methylellipticinium acetate, indazole-pyridine A-443654, an isoform-specific allosteric kinase inhibitor, or a PI3K/AKT/mTOR inhibitor.

93. The kit of embodiments 91 or 92, wherein the AKT inhibitor includes BEZ235, BKM120, Everolimus, MK-2206 dichloride, Pictilisib, LY294002, CAL-101, PI-3065, HS-173, PI-103, NU7441, TGX-221, 10-87114, Wortmannin, XL147, ZSTK474, BYL719, AS-605240, PIK-75, 3-methyladenine, A66, SAR245409, PIK-93, GSK2126458, PIK-90, PF-04691502, AZD6482, Apitolisib, GSK1059615, Duvelisib, Gedatolisib, TG100-1 15, AS-252424, BGT226, CUDC-907, PIK-294, AS-604850, GSK2636771, BAY80-6946, YM201636, CH5132799, CAY10505, rapamycin, PIK-293, GSK690693, Ipatasertib (GDC-0068), Capivasertib (AZD5363), PF-04691502, AT7867, Triciribine (NSC 154020), CCT128930, A-674563, PHT-427, Miransertib (ARQ 092) HCl, Uprosertib (GSK2141795), Afuresertib (GSK2110183), AT13148, Miltefosine, Honokiol (NSC 293100), TIC10 Analogue, Deguelin, or TIC10 (ONC201).

94. The kit of any of embodiments 91-93, wherein the AKT inhibitor includes an antisense oligonucleotide.

95. The kit of embodiment 94, wherein the antisense oligonucleotide has a sequence as set forth in SEQ ID NO: 1, SEQ ID NO: 2, SEQ ID NO: 3, SEQ ID NO: 4, SEQ ID NO: 5, SEQ ID NO: 6, or SEQ ID NO: 7 or a sequence having at least 95% sequence identity to the sequence as set forth in SEQ ID NO: 1, SEQ ID NO: 2, SEQ ID NO: 3, SEQ ID NO: 4, SEQ ID NO: 5, SEQ ID NO: 6, or SEQ ID NO: 7.

96. The kit of any of embodiments 91-95, wherein the anthracycline includes a compound of Formula I:

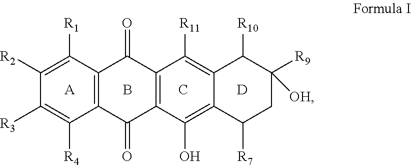

wherein:
R₁, R₂, R₃, R₄, R₇, R₉, R₁₀, R₁₁ are each independently selected in each instance from a group consisting of an organic substituent, an inorganic substituent, an open-chain substituent, a branched substituent, a cyclic substituent, or any combination thereof.

97. The kit of embodiment 96, wherein the R₁, R₂, R₃, R₄, R₇, R₉, R₁₀, R₁₁ are each independently selected in each instance from a group consisting of a hydrogen, a hydroxyl group, a methoxy group, an acetyl group, a quinone, or a phenolic group.

98. The kit of embodiments 96 or 97, wherein R₇ includes a mono- or multisaccharide substitution.

99. The kit of any of embodiments 91-98, wherein the anthracycline includes aclarubicin, aclacinomycin A, adriamycin, aklavin, AN-7A, AN-7B, AN-7D-apoprotein, auromomycin, carminomycin, cinerubin A, cinerubin B, cosmomycin A, dactinomycin, daunomycin, daunorubicin, dimethyl-doxorubicin, ditrisarubicin A, ditrisarubicin B, ditrisarubicin C, doxorubicin, epirubicin, galirubin A, galirubin B, galirubin D, galirubin S, HBW-6(B), HBW-6(A), idarubicin, MA 144-G1, MS 144-G2, MA 144-L, MA 144-M1, MA 144-M2, MA 144-N1, MA 144-S1, MA 144-S2, MA 144-U1, MA 144-U2, MA 144-Y, macromomycin, marcellomycin, mitoxantrone, musettamycin, NCS-apoprotein, nemorubicin, neocarzinostatin, nogalamycin, pixantrone, plicamycin, pyrromycin, requinomycin, rhodomycin A, rhodomycin X, rhodomycin Y, rubidazone, rubidomycin, sabarubicin, steffimycin, trypanomycin, valrubicin, violamycin, or γ-rhodomycin Y.

100. The kit of any of embodiments 90-99, wherein the methylation status includes global methylation status.

101. The kit of any of embodiments 90-100, wherein the reagents to detect methylation status include DNA-fragmenting enzymes.

102. The kit of any of embodiments 90-101, further including bisulfite.

103. The kit of any of embodiments 90-102, wherein the reagents to detect methylation status include a replication enzyme.

104. The kit of embodiment 103, wherein the replication enzyme is a thermostable DNA polymerase.

105. The kit of any of embodiments 90-104, wherein the reagents to detect methylation status include deoxynucleotide triphosphates (dNTPs).

106. The kit of any of embodiments 90-105, wherein the reagents to detect methylation status include probes.

107. The kit of embodiment 106, wherein each probe includes a detectable label.

108. The kit of embodiment 107, wherein the detectable label is a fluorescent label, dye, radioactive isotope, enzyme, magnetic bead, or biotin.

109. The kit of embodiments 107 or 108, wherein the detectable label is a fluorescent label.

110. The kit of any of embodiments 90-109, further including a reference level.

111. The kit of embodiment 110, wherein the reference level is derived from a population of subjects with DHMC.

112. The kit of embodiment 110, wherein the reference level is derived from a population of subjects without DHMC.

113. The kit of any of embodiments 90-112, wherein the kit further includes reagents to detect other biomarkers.

114. The kit of any of embodiments 90-113, wherein the kit can be used with other kits for detecting biomarkers.

115. The kit of any of embodiments 90-114, further including a sample collector.

116. The kit of any of embodiments 90-115, further including a DNA methyltransferase (DNMT) inhibitor.

117. The kit of embodiment 116, wherein the DNMT inhibitor includes decitabine, GSK-3484862, azacytidine, hydralazine, procaine, MG98, or zebularine.

118. The kit of any of embodiments 90-117, further including a polycomb repressive complex 2 (PRC2) inhibitor.

119. The kit of embodiment 118, wherein the PRC2 inhibitor includes enhancer zeste homolog 2 (EZH2) inhibitor or an embryonic ectoderm development (EED) inhibitor.

120. The kit of embodiment 119, wherein the EZH2 inhibitor includes GSK126, MAK683, EPZ6438, EPZ005687, CPI-1205, PF-06821497, SHR2554, UNC1999, valemetostat tosylate, CPI-169, UNC6852, or A-395.

121. The kit of embodiments 119 or 120, wherein the EED inhibitor includes EED226, ORIC-944, LG1980, MAK683, A-395, BR-001, EEDi-5285, EEDi-5273, FTX-6058, EED162, EED709, or Jarid2114-118-K116 me3.

122. The kit of any of embodiments 90-121, further including a chemotherapeutic agent.

### (ix) Experimental Example

Targeting vulnerabilities arising from global DNA hypomethylation in cancer. DNA methylation alterations are a universal feature of cancer. In addition to site specific gain of DNA methylation (hypermethylation), a global loss of methylation (hypomethylation) was noted in most cancer genomes. Whereas numerous studies have focused on the role of hypermethylation in disease progression, less is known about the biology of hypomethylation in cancer. Recently, tumors across all major cancer types that are characterized by severe loss of DNA methylation were identified. The data demonstrates that these DNA hypomethylated cancers (hereafter DHMCs) show distinct alterations influencing gene expression and tumor microenvironment composition.

Since DNA methylation is critical for genome organization and gene expression, severe global hypomethylation would likely result in distinct epigenomic and genomic alterations. Common molecular changes associated with DNA hypomethylation was therefore investigated to develop novel therapeutic approaches targeting unique vulnerabilities arising in DHMCs.

To delineate the biology of DHMCs, methylation changes were determined with validated orthogonal methods in large representative cohorts of patient samples (The Cancer Genome Atlas (TCGA), University of Washington rapid autopsy cohorts), patient derived xenografts and cancer cell lines. The patterns of common molecular alterations were assessed by performing genome wide epigenome mapping experiments and by defining common driver gene events in DHMCs. Tumor cell intrinsic therapeutic vulnerabilities were further determined in broad pharmacologic and CRISPR-Cas9 genomic screens.

It was observed that DNA hypomethylation is a relatively common feature of solid tumors affecting overall 15-20% of all cancers, with highest rates observed in urothelial, melanoma, hepatocellular and lung and head and neck squamous cell carcinoma. In in vivo tumor models of DHMCs, distinct shifts in epigenetic states were observed upon DNA hypomethylation.

This is the first study to show druggable vulnerabilities arising from DNA hypomethylation. Collectively, these studies define a novel epigenetic subtype of cancer, determine its clinical and molecular features, and pave the way for new targeted therapies.

### (x) Closing Paragraphs

The nucleic acid and amino acid sequences provided herein are shown using letter abbreviations for nucleotide bases and amino acid residues, as defined in 37 C.F.R. § 1.831-1.835 and set forth in WIPO Standard ST.26 (implemented on Jul. 1, 2022). Only one strand of each nucleic acid sequence is shown, but the complementary strand is understood as included in embodiments where it would be appropriate.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically-significant reduction in destruction of DHMC cells.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Unless otherwise indicated, the practice of the present disclosure can employ conventional techniques of immunology, molecular biology, microbiology, cell biology and recombinant DNA. These methods are described in the following publications. See, e.g., Green and Sambrook, Molecular Cloning: A Laboratory Manual, 4nd Edition (2012); F. M. Ausubel, et al. eds., Current Protocols in Molecular Biology, (2003); the series Methods In Enzymology (Academic Press, Inc.); Behlke, et al., Polymerase Chain Reaction: Theory and Technology (2019); Greenfield, ed. Antibodies, A Laboratory Manual, Second Edition (2014); and Capes-Davis and R. I. Freshney, eds. Freshney's Culture of Animal Cells 8th Edition (2021).

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of ordinary skill in the art, such as the Oxford Dictionary of Biochemistry and Molecular Biology (Ed. Anthony Smith, Oxford University Press, Oxford, 2004).

1-122. (canceled)

123. A method comprising:
identifying a subject having DNA hypomethylated cancer (DHMC); and
administering to the subject a therapeutically effective amount of a DHMC-sensitive compound
wherein the DHMC cancer comprises an adrenal cancer, astrocytoma, bladder cancer, breast cancer, colon cancer, chordoma, choroid plexus carcinoma, choroid plexus papilloma, endometrial cancer, ependymoma, extragonadal germ cell tumor, glioblastoma, head and neck cancer, hepatocellular carcinoma, intestinal cancer, kidney cancer, leukemia, lung cancer, lymphoma, leukemia, malignant rhabdoid tumor, medulloblastoma, melanoma, meningioma, mesothelioma, multiple myeloma, neuroglial tumor, pancreatic cancer, oligodendroglioma, oligoastrocytoma, ovarian cancer, pineoblastoma, prostate cancer, renal cell carcinoma, renal medullary carcinoma, sarcoma, stem cell cancer, stomach cancer, testicular cancer, urothelial cancer, or uterine cancer.

124. The method of claim 123, wherein the DHMC-sensitive compound comprises an AKT inhibitor, an anthracycline, or an antisense oligonucleotide,
wherein the AKT inhibitor comprises BEZ235, BKM120, Everolimus, MK-2206 dichloride, Pictilisib, LY294002, CAL-101, PI-3065, HS-173, PI-103, NU7441, TGX-221, 10-87114, Wortmannin, XL147, ZSTK474, BYL719, AS-605240, PIK-75, 3-methyladenine, A66, SAR245409, PIK-93, GSK2126458, PIK-90, PF-04691502, AZD6482, Apitolisib, GSK1059615, Duvelisib, Gedatolisib, TG100-1 15, AS-252424, BGT226, CUDC-907, PIK-294, AS-604850, GSK2636771, BAY80-6946, YM201636, CH5132799, CAY10505, rapamycin, PIK-293, GSK690693, Ipatasertib (GDC-0068), Capivasertib (AZD5363), PF-04691502, AT7867, Triciribine (NSC 154020), CCT128930, A-674563, PHT-427, Miransertib (ARQ 092) HCl, Uprosertib (GSK2141795), Afuresertib (GSK2110183), AT13148, Miltefosine, Honokiol (NSC 293100), TIC10 Analogue, Deguelin, or TIC10 (ONC201);
wherein the anthracycline comprises aclarubicin, aclacinomycin A, adriamycin, aklavin, AN-7A, AN-7B, AN-7D-apoprotein, auromomycin, carminomycin, cinerubin A, cinerubin B, cosmomycin A, dactinomycin, daunomycin, daunorubicin, dimethyl-doxorubicin, ditrisarubicin A, ditrisarubicin B, ditrisarubicin C, doxorubicin, epirubicin, galirubin A, galirubin B, galirubin D, galirubin S, HBW-6(B), HBW-6(A), idarubicin, MA 144-G1, MS 144-G2, MA 144-L, MA 144-M1, MA 144-M2, MA 144-N1, MA 144-S1, MA 144-S2, MA 144-U1, MA 144-U2, MA 144-Y, macromomycin, marcellomycin, mitoxantrone, musettamycin, NCS-apoprotein, nemorubicin, neocarzinostatin, nogalamycin, pixantrone, plicamycin, pyrromycin, requinomycin, rhodomycin A, rhodomycin X, rhodomycin Y, rubidazone, rubidomycin, sabarubicin, steffimycin, trypanomycin, valrubicin, violamycin, or γ-rhodomycin Y; and/or
wherein the antisense oligonucleotide comprises a sequence as set forth in SEQ ID NO: 1, SEQ ID NO: 2, SEQ ID NO: 3, SEQ ID NO: 4, SEQ ID NO: 5, SEQ ID NO: 6, or SEQ ID NO: 7 or a sequence having at least 95% sequence identity to the sequence as set forth in SEQ ID NO: 1, SEQ ID NO: 2, SEQ ID NO: 3, SEQ ID NO: 4, SEQ ID NO: 5, SEQ ID NO: 6, or SEQ ID NO: 7.

125. The method of claim 123, further comprising administering to the subject a DNA methyltransferase (DNMT) inhibitor, a polycomb repressive complex 2 (PRC2) inhibitor, chemotherapy, and/or radiation,
wherein the DNMT inhibitor comprises decitabine, GSK-3484862, azacytidine, hydralazine, procaine, MG98, or zebularine; and/or
wherein the PRC2 inhibitor comprises GSK126, MAK683, EPZ6438, EPZ005687, CPI-1205, PF-06821497, SHR2554, UNC1999, valemetostat tosylate, CPI-169, UNC6852, A-395, EED226, ORIC-944, LG1980, MAK683, A-395, BR-001, EEDi-5285, EEDi-5273, FTX-6058, EED162, EED709, or Jarid2114-118-K116 me3.

126. The method of claim 123, wherein the administering comprises injection, infusion, perfusion, lavage, or ingestion.

127. A method comprising:
obtaining a sample derived from a subject;
processing the sample to determine a DNA methylation level of the sample; and
determining that the methylation level is hypomethylated and that the subject will be sensitive to treatment with a DNA hypomethylate cancer (DHMC)-sensitive compound.

128. The method of claim 127, wherein the subject has cancer.

129. The method of claim 128, wherein the cancer comprises an adrenal cancer, astrocytoma, bladder cancer, breast cancer, colon cancer, chordoma, choroid plexus carcinoma, choroid plexus papilloma, endometrial cancer, ependymoma, extragonadal germ cell tumor, glioblastoma, head and neck cancer, hepatocellular carcinoma, intestinal cancer, kidney cancer, leukemia, lung cancer, lymphoma, leukemia, malignant rhabdoid tumor, medulloblastoma, melanoma, meningioma, mesothelioma, multiple myeloma, neuroglial tumor, pancreatic cancer, oligodendroglioma, oligoastrocytoma, ovarian cancer, pineoblastoma, prostate cancer, renal cell carcinoma, renal medullo carcinoma, sarcoma, stem cell cancer, stomach cancer, testicular cancer, urothelial cancer, or uterine cancer.

130. The method of claim 127, wherein the sample comprises a tumor biopsy sample or a liquid sample comprising blood, plasma, cerebrospinal fluid, sputum, stool, urine, lymphatic fluid, or saliva.

131. The method of claim 127, wherein the methylation level is a global methylation level.

132. The method of claim 127, further comprising generating a report comprising data indicative of the methylation level.

133. The method of claim 132, wherein the report provides a recommendation treatment to administer to the subject.

134. The method of claim 133, wherein the recommended treatment comprises administering the DHMC-sensitive compound.

135. The method of claim 134, wherein the recommended DHMC-sensitive compound comprises an AKT inhibitor, an anthracycline, or an antisense oligonucleotide, wherein the AKT inhibitor comprises BEZ235, BKM120, Everolimus, MK-2206 dichloride, Pictilisib, LY294002, CAL-101, PI-3065, HS-173, PI-103, NU7441, TGX-221, 10-87114, Wortmannin, XL147, ZSTK474, BYL719, AS-605240, PIK-75, 3-methyladenine, A66, SAR245409, PIK-93, GSK2126458, PIK-90, PF-04691502, AZD6482, Apitolisib, GSK1059615, Duvelisib, Gedatolisib, TG100-1 15, AS-252424, BGT226, CUDC-907, PIK-294, AS-604850, GSK2636771, BAY80-6946, YM201636, CH5132799, CAY10505, rapamycin, PIK-293, GSK690693, Ipatasertib (GDC-0068), Capivasertib (AZD5363), PF-04691502, AT7867, Triciribine (NSC 154020), CCT128930, A-674563, PHT-427, Miransertib (ARQ 092) HCl, Uprosertib (GSK2141795), Afuresertib (GSK2110183), AT13148, Miltefosine, Honokiol (NSC 293100), TIC10 Analogue, Deguelin, or TIC10 (ONC201);
wherein the anthracycline comprises aclarubicin, aclacinomycin A, adriamycin, aklavin, AN-7A, AN-7B, AN-7D-apoprotein, auromomycin, carminomycin, cinerubin A, cinerubin B, cosmomycin A, dactinomycin, daunomycin, daunorubicin, dimethyl-doxorubicin, ditrisarubicin A, ditrisarubicin B, ditrisarubicin C, doxorubicin, epirubicin, galirubin A, galirubin B, galirubin D, galirubin S, HBW-6(B), HBW-6(A), idarubicin, MA 144-G1, MS 144-G2, MA 144-L, MA 144-M1, MA 144-M2, MA 144-N1, MA 144-S1, MA 144-S2, MA 144-U1, MA 144-U2, MA 144-Y, macromomycin, marcellomycin, mitoxantrone, musettamycin, NCS-apoprotein, nemorubicin, neocarzinostatin, nogalamycin, pixantrone, plicamycin, pyrromycin, requinomycin, rhodomycin A, rhodomycin X, rhodomycin Y, rubidazone, rubidomycin, sabarubicin, steffimycin, trypanomycin, valrubicin, violamycin, or γ-rhodomycin Y; and/or
wherein the antisense oligonucleotide comprises a sequence as set forth in SEQ ID NO: 1, SEQ ID NO: 2, SEQ ID NO: 3, SEQ ID NO: 4, SEQ ID NO: 5, SEQ ID NO: 6, or SEQ ID NO: 7 or a sequence having at least 95% sequence identity to the sequence as set forth in SEQ ID NO: 1, SEQ ID NO: 2, SEQ ID NO: 3, SEQ ID NO: 4, SEQ ID NO: 5, SEQ ID NO: 6, or SEQ ID NO: 7.

136. The method of claim 134, wherein the recommended treatment further comprises administering a DNA methyltransferase (DNMT) inhibitor, a polycomb repressive complex 2 (PRC2) inhibitor, chemotherapy, or radiation,
wherein the DNMT inhibitor comprises decitabine, GSK-3484862, azacytidine, hydralazine, procaine, MG98, or zebularine; and/or
wherein the PRC2 inhibitor comprises GSK126, MAK683, EPZ6438, EPZ005687, CPI-1205, PF-06821497, SHR2554, UNC1999, valemetostat tosylate, CPI-169, UNC6852, A-395, EED226, ORIC-944, LG1980, MAK683, A-395, BR-001, EEDi-5285, EEDi-5273, FTX-6058, EED162, EED709, or Jarid2114-118-K116 me3.

137. The method of claim 132, further comprising transmitting the report to an external device.

138. A kit comprising a DNA hypomethylated cancer (DHMC)-sensitive compound and reagents to detect methylation status of DNA.

139. The kit of claim 138, wherein the recommended DHMC-sensitive compound comprises an AKT inhibitor, an anthracycline, or an antisense oligonucleotide, wherein the AKT inhibitor comprises BEZ235, BKM120, Everolimus, MK-2206 dichloride, Pictilisib, LY294002, CAL-101, PI-3065, HS-173, PI-103, NU7441, TGX-221, 10-87114, Wortmannin, XL147, ZSTK474, BYL719, AS-605240, PIK-75, 3-methyladenine, A66, SAR245409, PIK-93, GSK2126458, PIK-90, PF-04691502, AZD6482, Apitolisib, GSK1059615, Duvelisib, Gedatolisib, TG100-1 15, AS-252424, BGT226, CUDC-907, PIK-294, AS-604850, GSK2636771, BAY80-6946, YM201636, CH5132799, CAY10505, rapamycin, PIK-293, GSK690693, Ipatasertib (GDC-0068), Capivasertib (AZD5363), PF-04691502, AT7867, Triciribine (NSC 154020), CCT128930, A-674563, PHT-427, Miransertib (ARQ 092) HCl, Uprosertib (GSK2141795), Afuresertib (GSK2110183), AT13148, Miltefosine, Honokiol (NSC 293100), TIC10 Analogue, Deguelin, or TIC10 (ONC201);
wherein the anthracycline comprises aclarubicin, aclacinomycin A, adriamycin, aklavin, AN-7A, AN-7B, AN-7D-apoprotein, auromomycin, carminomycin, cinerubin A, cinerubin B, cosmomycin A, dactinomycin, daunomycin, daunorubicin, dimethyl-doxorubicin, ditrisarubicin A, ditrisarubicin B, ditrisarubicin C, doxorubicin, epirubicin, galirubin A, galirubin B, galirubin D, galirubin S, HBW-6(B), HBW-6(A), idarubicin, MA 144-G1, MS 144-G2, MA 144-L, MA 144-M1, MA 144-M2, MA 144-N1, MA 144-S1, MA 144-S2, MA 144-U1, MA 144-U2, MA 144-Y, macromomycin, marcellomycin, mitoxantrone, musettamycin, NCS-apoprotein, nemorubicin, neocarzinostatin, nogalamycin, pixantrone, plicamycin, pyrromycin, requinomycin, rhodomycin A, rhodomycin X, rhodomycin Y, rubidazone, rubidomycin, sabarubicin, steffimycin, trypanomycin, valrubicin, violamycin, or γ-rhodomycin Y; and/or
wherein the antisense oligonucleotide comprises a sequence as set forth in SEQ ID NO: 1, SEQ ID NO: 2, SEQ ID NO: 3, SEQ ID NO: 4, SEQ ID NO: 5, SEQ ID NO: 6, or SEQ ID NO: 7 or a sequence having at least 95% sequence identity to the sequence as set forth in SEQ ID NO: 1, SEQ ID NO: 2, SEQ ID NO: 3, SEQ ID NO: 4, SEQ ID NO: 5, SEQ ID NO: 6, or SEQ ID NO: 7.

140. The kit of claim 138, wherein the kit comprises DNA-fragmenting enzymes, a replication enzyme, deoxynucleotide triphosphates (dNTPs), a detectable label, and a reference level derived from a population of subjects with DHMC or from a population of subjects without DHMC.

141. The kit of claim 138, further comprising a DNA methyltransferase (DNMT) inhibitor or a polycomb repressive complex 2 (PRC2) inhibitor
wherein the DNMT inhibitor comprises decitabine, GSK-3484862, azacytidine, hydralazine, procaine, MG98, or zebularine; and/or
wherein the PRC2 inhibitor comprises GSK126, MAK683, EPZ6438, EPZ005687, CPI-1205,
EED226, ORIC-944, LG1980, MAK683, A-395, BR-001, EEDi-5285, EEDi-5273, FTX-6058, EED162, EED709, or Jarid2114-118-K116 me3.

142. The kit of claim 138, further comprising a chemotherapeutic agent.
